(19) United States
(12) Patent Application Publication
(10) Pub. No.: US 2026/0242490 A1
(43) Pub. Date: Aug. 20, 2026

(54) METHODS OF TREATING NAFLD AND NASH

(71) Applicant: CytoDyn Inc., Vancouver, WA (US)

(72) Inventors: Scott KELLY, Vancouver, WA (US); Christopher RECKNOR, Vancouver, WA (US)

(21) Appl. No.: 18/876,791

(22) PCT Filed: Jun. 21, 2023
(86) PCT No.: PCT/US2023/025878

§ 371 Date: Dec. 19, 2024

Related U.S. Application Data
(60) Provisional application No. 63/354,664, filed on Jun. 22, 2022.

(51) Int. Cl. C07K 16/28; A61K 9/00; A61K 39/00; A61P 1/16

(52) U.S. Cl. CPC C07K 16/2866; A61K 9/0019; A61P 1/16; A61K 2039/505; A61K 2039/545; C07K 2317/24

(57) ABSTRACT

The present disclosure provides CCR5 binding agents that are useful in preventing or treating CCR5 and CCR2 dysregulation and resulting conditions and symptoms thereof, including NASH and NAFLD. The present disclosure provides a method of treating or preventing nonalcoholic fatty liver disease (NAFLD) or nonalcoholic steatohepatitis (NASH) in a patient by administering to the patient an amount of leronlimab effective to treat or prevent NAFLD or NASH in the form of a weekly injection.

### TECHNICAL FIELD

The present disclosure relates to a method of treating or preventing nonalcoholic fatty liver disease NAFLD, particularly nonalcoholic steatohepatitis (NASH) by administering an anti-CCR5 antibody or antigen binding fragment, such as leronlimab or an antigen binding fragment thereof.

### BACKGROUND

The roles of C-C Motif Chemokine Ligand 5 (CCL5) (also known as (regulated on activation, normal T cell expressed and secreted (RANTES)) in binding to CCR5 and of C-C Motif Chemokine Ligand 2 in binding to CCR2 and subsequent cellular signaling have been well-characterized. Signaling associated CCR5/RANTES binding supports chemotaxis of pro-inflammatory cells, whereas CCR2/CCL2 signaling via IL-10 supports chemotaxis of immune suppressive cells such as M2 monocytes, myeloid derived suppressors and dendritic cells which regulate/prevent the transition from an innate (Th0) to acquired (Th1/Th2) immune response. CCR2 signaling via CCL2 has been very well characterized and has been associated with creating pro-tumor environment.

### SUMMARY

The present disclosure provides a method of treating or preventing nonalcoholic fatty liver disease (NAFLD) or nonalcoholic steatohepatitis (NASH) in a patient by administering to the patient an amount of leronlimab effective to treat or prevent NAFLD or NASH.

The method further provides:
wherein the leronlimab is administered by injection;
wherein the leronlimab is administered weekly;
wherein the leronlimab is administered in an amount effective to also treat or prevent NASH-related liver fibrosis;
wherein the leronlimab is administered in a 350 mg dose;
wherein the leronlimab is administered in a 525 mg dose;
wherein the leronlimab is administered in a 700 mg dose;
wherein the patient is evaluated for CCR5 haplotye, and, if the patient has a CCR5 haplotype not associated with increased CCR5 cell surface expression, the patient is administered 350 mg of leronlimab weekly;
wherein the patient is evaluated for CCR5 haplotye, and, if the patient has a CCR5 haplotype not associated with increased CCR5 cell surface expression, the patient is administered 525 mg of leronlimab weekly;
wherein the CCR5 haplotype not associated with increased CCR5 cell surface expression does not comprise HHE or HHG;
wherein the patient is evaluated for CCR5 haplotye, and, if the patient has a CCR5 haplotype associated with increased CCR5 cell surface expression, the patient is administered 525 mg of leronlimab weekly;
wherein the patient is evaluated for CCR5 haplotype, and, if the patient has a CCR5 haplotype associated with increased CCR5 cell surface expression, the patient is administered 700 mg of leronlimab weekly;
wherein the CCR5 haplotype associated with increased CCR5 cell surface expression comprises HHE or HHG;
wherein, subsequent to administration of at least one dose of leronlimab, the level of a biomarker indicating liver function or inflammation is measured, and the dose or dosing frequency of leronlimab is adjusted if the level is not changed by a preset amount as compared to a baseline level or prior level, or if the level is not above or below a preset value;
wherein the biomarker is one or more of RANTES, CCL2, CCL3, CCL11, CCL18, VCAM, and EN RAGE;
wherein the biomarker is RANTES and, if the serum level is not sufficiently low to have no clinical inflammatory effect on NASH, the dose or frequency of administration of leronlimab is increased;
wherein the dose is increased from 350 mg weekly to 700 mg weekly;
wherein, subsequent to administration of at least one dose of leronlimab, the level of an MRI indicator of NASH is measured, and the dose or dosing frequency of leronlimab is adjusted if the level is not changed by a preset amount as compared to a baseline level or prior level, or if the level is not above or below a preset value;
wherein the MRI indicator of NASH is PDFF or cT1;
wherein, if the PDFF or cT1 is not below a preset level or decreased as compared to a prior measurement for the patient, the dose of leronlimab is increased from 350 mg weekly to 700 mg weekly;
and any compatible combinations thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The disclosure may be better understood through reference to the following figures.

FIG. 1A is a set of photomicrographs of frozen liver sections from mice fed a high fat, high cholesterol diet and then treated with leronlimab or a generic IgG. Sections are stained with Oil Red O and are at 20× magnification.

FIG. 1B is a graph showing the percent of image in an entire region of interest that is positive for Oil Red O from the images of FIG. 1A.

FIG. 2 is a set of graphs showing demographics of participants in a clinical trial as described herein in Example 2.

FIG. 3 is a graph showing adverse events over the clinical trial of Example 2 based on treatment type.

FIG. 4 is a graph showing the total number of adverse events of FIG. 3 per treatment type.

FIG. 5 is a Z-score heatmap showing change in cytokine markers from baseline as measured at the beginning and end of the clinical trial of Example 2 by treatment type.

FIG. 6A is a set of graphs showing change from baseline in cT1 levels (left panel) and PDFF levels (right panel) in patients in the clinical trial of Example 2, as measured by magnetic resonance imagining (MRI).

FIG. 6B is a set of representative micrographs of the type used to obtain the data in FIG. 6A.

FIG. 7 illustrates a likely mechanism of action for effects of leronlimab stabilization of CCR5 on the cell surface.

FIG. 8 illustrates a likely mechanism of action for the effects of leronlimab on VCAM.

FIG. 9 illustrates a likely mechanism of action for the effects of leronlimab on inflammation biomarkers.

FIG. 10 illustrates a likely mechanism of action for the effects of leronlimab on T cell balance and TH response.

FIG. 11 illustrates a likely mechanism of action for the effects of leronlimab on cardiovascular markers.

FIG. 12 illustrates a likely mechanism of action for the effects of leronlimab in patients with CCR5 overexpression.

### DETAILED DESCRIPTION

The present disclosure relates to a method of treating NAFLD, particularly NASH, and related symptoms and resulting further disorders by i) treating CCR5 and CCR2 dysregulation, ii) administering and anti-CCR5 antibody or antigen binding fragment, such as leronlimab or an antigen binding fragment, or iii) both.

Prior to setting forth this disclosure in more detail, it may be helpful to an understanding thereof to provide definitions of certain terms to be used herein. Unless defined otherwise, all technical and scientific terms used herein have the same meaning as is commonly understood by one of skill in the art to which this invention belongs. Additional definitions are set forth throughout this disclosure.

In the present description, the term “about” means±20% of the indicated range, value, or structure, unless otherwise indicated. The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof. Any ranges provided herein include all the values and narrower ranges in the ranges.

As used herein, “chemokine” refers to a low-molecular weight cytokine that can stimulate recruitment of leukocytes. Chemokines have cysteine residues in conserved locations that are key to forming their 3-dimensional shape. Chemokines may be classified into four main subfamilies: Cys-Cys (C-C), Cys-X-Cys (CXC), CX3C, and XC depending on the spacing of their first two amino terminal cysteine residues. Chemokines may also be grouped according to their function, such as whether they are inflammatory or homeostatic. There are 47 known chemokines, including but not limited to CCL5 (also known as RANTES), MIP-1a, MIP-10, or SDF-1, or another chemokine which has similar activity.

As used herein, “C-C chemokine receptor 5,” also known as “CCR5” or “CD195” refers to a G protein-coupled receptor expressed on lymphocytes (e.g., NK cells, B cells, T cells), monocytes, dendritic cells, eosinophils, and microglia, which functions as a chemokine receptor for the C-C chemokine group. CCR5's cognate ligands include CCL3, CCL4, CCL3L1, and CCL5. In some embodiments, CCR5 refers to human CCR5. In some embodiments, CCR5 refers to a protein having an amino acid sequence provided in NCBI Reference Sequence: NP_000570.1 (SEQ ID NO:15).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

As used herein, “protein” or “polypeptide” as used herein refers to a compound made up of amino acid residues that are covalently linked by peptide bonds. The term “protein” may be synonymous with the term “polypeptide” or may refer, in addition, to a complex of two or more polypeptides. A polypeptide may further contain other components (e.g., covalently bound), such as a tag, a label, a bioactive molecule, or any combination thereof. In certain embodiments, a polypeptide may be a fragment. As used herein, a “fragment” means a polypeptide that is lacking one or more amino acids that are found in a reference sequence. A fragment can comprise a binding domain, antigen, or epitope found in a reference sequence. A fragment of a reference polypeptide can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of amino acids of the amino acid sequence of the reference sequence.

As described herein, a “variant” polypeptide species has one or more non-natural amino acids, one or more amino acid substitutions, one or more amino acid insertions, one or more amino acid deletions, or any combination thereof at one or more sites relative to a reference polypeptide as presented herein. In certain embodiments, “variant” means a polypeptide having a substantially similar activity (e.g., enzymatic function, immunogenicity) or structure relative to a reference polypeptide). A variant of a reference polypeptide can have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the reference polypeptide as determined by sequence alignment programs and parameters known in the art. The variant can result from, for example, a genetic polymorphism or human manipulation. Conservative substitutions of amino acids are well known and may occur naturally or may be introduced when a protein is recombinantly produced. Amino acid substitutions, deletions, and additions may be introduced into a protein using mutagenesis methods known in the art (see, e.g., Sambrook et al., Molecular Cloning: A Laboratory Manual, 3d ed., Cold Spring Harbor Laboratory Press, NY, 2001). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare polypeptide variants (see, e.g., Sambrook et al., supra).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. The algorithm used herein for determining percent sequence identity and sequence similarity is the BLAST 2.0 algorithm, as described in Altschul et al. “Gapped BLAST and PSI-BLAST: a new generation of protein database search programs,” Nucleic Acids Res. 2007, 25, 3389-3402. Within the context of this disclosure, it will be understood that where sequence analysis software is used for analysis, the results of the analysis are based on the “default values” of the program referenced. “Default values” mean any set of values or parameters which originally load with the software when first initialized.

As used herein, a “fusion protein” comprises a single chain polypeptide having at least two distinct domains, wherein the domains are not naturally found together in a protein. A nucleic acid molecule encoding a fusion protein may be constructed using PCR, recombinantly engineered, or the like, or such fusion proteins can be made synthetically. A fusion protein may further contain other components (e.g., covalently bound), such as a tag, linker, transduction marker, or bioactive molecule.

A “nucleic acid molecule” or “polynucleotide” refers to a polymeric compound containing nucleotides that are covalently linked by 3′-5′ phosphodiester bonds. Nucleic acid molecules include polyribonucleic acid (RNA), polydeoxyribonucleic acid (DNA), which includes genomic DNA, mitochondrial DNA, cDNA, or vector DNA. A nucleic acid molecule may be double stranded or single stranded, and if single stranded, may be the coding strand or non-coding (anti-sense strand). A nucleic acid molecule may contain natural subunits or non-natural subunits. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of the polynucleotides of this disclosure are also contemplated. Variant polynucleotides are at least 80%, 85%, 90%, 95%, 99%, or 99.9% identical to a reference polynucleotide as described herein, or that hybridizes to a reference polynucleotide of defined sequence under stringent hybridization conditions of 0.015M sodium chloride, 0.0015M sodium citrate at about 65°−68° C. or 0.015M sodium chloride, 0.0015M sodium citrate, and 50% formamide at about 42° C. The polynucleotide variants retain the capacity to encode an immunoglobulin-like binding protein or antigen-binding fragment thereof having the functionality described herein.

The term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the co-existing materials in the natural system, is isolated. Such polynucleotide could be part of a vector and/or such polynucleotide or polypeptide could be part of a composition (e.g., a cell lysate), and still be isolated in that such vector or composition is not part of the natural environment for the nucleic acid or polypeptide.

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein, or other activity encoded by an exogenous nucleic acid molecule introduced into the host cell, even if the host cell encodes a homologous protein or activity.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

As used herein, the term “expression”, refers to the process by which a polypeptide is produced based on the encoding sequence of a nucleic acid molecule, such as a gene. The process may include transcription, post-transcriptional control, post-transcriptional modification, translation, post-translational control, posttranslational modification, or any combination thereof. An expressed nucleic acid molecule is typically operably linked to an expression control sequence (e.g., a promoter).

As used herein, the term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

As used herein, the term “host” refers to a cell (e.g., T cell, Chinese Hamster Ovary (CHO) cell, HEK293 cell, B cell, or the like) or microorganism targeted for genetic modification with a heterologous nucleic acid molecule to produce a polypeptide of interest (e.g., a CCR5 antibody of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous BCR).

As described herein, more than one heterologous nucleic acid molecule can be introduced into a host cell as separate nucleic acid molecules, as a plurality of individually controlled genes, as a polycistronic nucleic acid molecule (e.g., a heavy chain and a light chain of an antibody), as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “introduced” in the context of inserting a nucleic acid sequence into a cell, means “transfection”, or “transformation” or “transduction” and includes reference to the incorporation of a nucleic acid sequence into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

“Treat” or “treatment” or “ameliorate” refers to medical management of a disease, disorder, or condition of a patient (e.g., a human or non-human mammal, such as a primate, horse, cat, dog, goat, mouse, or rat). In general, an appropriate dose or treatment regimen comprising leronlimab is administered in an amount sufficient to elicit a therapeutic effect or therapeutic benefit. Therapeutic effect or therapeutic benefit includes improved clinical outcome; modulation of immune response to lessen, reduce, or dampen counterproductive inflammatory cytokine activity; modulation of immune response to normalize counterproductive inflammatory cytokine activity; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof. Patients who have been treated may also be referred to as “dosed,” whereas patients who have not been treated may be referred to as “non-dosed.”

A prophylactic treatment meant to “prevent” a disease or condition (e.g., coronavirus induced respiratory illness in a patient or patient) is a treatment administered to a patient who does not exhibit signs of a disease or exhibits only early signs, for the purpose of decreasing the risk of developing pathology or further advancement of the early disease. For example, if an individual at risk of developing a coronavirus induced respiratory illness is treated with the methods of the present disclosure and does not later develop coronavirus induced respiratory illness, then the disease has been prevented, at least over a period of time, in that individual. A prophylactic treatment can mean preventing recurrence of a disease or condition in a patient that has previously been treated for the disease or condition, e.g., by preventing relapse or recurrence of coronavirus induced respiratory illness.

A “therapeutically effective amount” or “effective amount” of leronlimab refers to an amount of leronlimab sufficient to result in a therapeutic effect, including improved clinical outcome; lessening or alleviation of symptoms associated with a disease; modulating immune response to lessen, reduce, or dampen counterproductive inflammatory cytokine activity; modulating immune response to normalize counterproductive inflammatory cytokine activity; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously.

As used herein, “relative reduction” or “relative risk reduction” refers to the percent reduction in a parameter (e.g. mortality, time to recovery) in the treated group (Y) compared to the control group (X). RR=1−(Y/X)×100%.

As used herein, “absolute reduction” or “absolute risk reduction” refers to the percent reduction between the control group (X) and the treatment group (Y). AR=X−Y.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian patient and generally recognized as safe or not causing a serious adverse event.

Additional definitions are provided in the sections below.

### Leronlimab

The present disclosure provides for use of leronlimab, or antigen binding fragment thereof, in treating or preventing NAFLD or NASH.

Terms understood by those in the art of antibody technology are each given the meaning acquired in the art, unless expressly defined differently herein. The term “antibody” refers to an intact antibody comprising at least two heavy (H) chains and two light (L) chains inter-connected by disulfide bonds, as well as any antigen-binding portion or fragment of an intact antibody, such as an scFv, Fab, or Fab′2 fragment, that has or retains the ability to bind to the antigen target molecule recognized by the intact antibody. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody). The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “VL” and “VH” refer to the variable binding region from an antibody light chain and an antibody heavy chain, respectively. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary amino acid sequence by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

Numbering of CDR and framework regions may be determined according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Pluckthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme.

In some embodiments, the present disclosure provides use of an anti-CCR5 antibody or antigen binding fragment thereof having a light chain variable region (VL) that is at least 70% identical to SEQ ID NO: 1, at least 75% identical to SEQ ID NO: 1, at least 80% identical to SEQ ID NO: 1, at least 85% identical to SEQ ID NO: 1, or at least 90% identical to SEQ ID NO: 1. In some embodiments, the present disclosure provides use of an anti-CCR5 antibody or antigen binding fragment thereof having a light chain variable antibody region that is 70%-100% identical to SEQ ID NO: 1, 75%-100% identical to SEQ ID NO: 1, 80%-100% identical to SEQ ID NO: 1, 85%-100% identical to SEQ ID NO: 1, 90%-100% identical to SEQ ID NO: 1 or 91%-100% identical to SEQ ID NO: 1.

In some embodiments, the present disclosure provides use of an anti-CCR5 antibody or antigen binding fragment thereof having a light chain variable region (VL) that is at least 70% identical to amino acids 20-131 of SEQ ID NO: 1, at least 75% identical to amino acids 20-131 of SEQ ID NO: 1, at least 80% identical to amino acids 20-131 of SEQ ID NO: 1, at least 85% identical to amino acids 20-131 of SEQ ID NO: 1, or at least 90% identical to amino acids 20-131 of SEQ ID NO: 1. In some embodiments, the present disclosure provides use of an anti-CCR5 antibody or antigen binding fragment thereof having a light chain variable antibody region that is 70%-100% identical to amino acids 20-131 of SEQ ID NO: 1, 75%-100% identical to amino acids 20-131 of SEQ ID NO: 1, 80%-100% identical to amino acids 20-131 of SEQ ID NO: 1, 85%-100% identical to amino acids 20-131 of SEQ ID NO: 1, 90%-100% identical to amino acids 20-131 of SEQ ID NO: 1 or 91%-100% identical to amino acids 20-131 of SEQ ID NO: 1.

In some embodiments, the present disclosure provides use of an anti-CCR5 antibody or antigen binding fragment thereof having a heavy chain variable region (VH) that is at least 70% identical to SEQ ID NO:3, at least 75% identical to SEQ ID NO:3, at least 80% identical to SEQ ID NO:3, at least 85% identical to SEQ ID NO:3, or at least 90% identical to SEQ ID NO:3. In some embodiments the present disclosure provides use of an anti-CCR5 antibody or antigen binding fragment thereof having a heavy chain antibody variable region that is 70%-100% identical to SEQ ID NO: 3, 75%-100% identical to SEQ ID NO: 3, 80%-100% identical to SEQ ID NO: 3, 85%-100% identical to SEQ ID NO: 3, 90%-100% identical to SEQ ID NO: 3, or 91%-100% identical to SEQ ID NO:3.

In some embodiments, the present disclosure provides use of an anti-CCR5 antibody or antigen binding fragment thereof having a heavy chain variable region (VH) that is at least 70% identical to amino acids 20-141 of SEQ ID NO:3, at least 75% identical to amino acids 20-141 of SEQ ID NO:3, at least 80% identical to amino acids 20-141 of SEQ ID NO:3, at least 85% identical to amino acids 20-141 of SEQ ID NO:3, or at least 90% identical to amino acids 20-141 of SEQ ID NO:3. In some embodiments the present disclosure provides use of an anti-CCR5 antibody or antigen binding fragment thereof having a heavy chain antibody variable region that is 70%-100% identical to amino acids 20-141 of SEQ ID NO: 3, 75%-100% identical to amino acids 20-141 of SEQ ID NO: 3, 80%-100% identical to amino acids 20-141 of SEQ ID NO: 3, 85%-100% identical to amino acids 20-141 of SEQ ID NO: 3, 90%-100% identical to amino acids 20-141 of SEQ ID NO: 3, or 91%-100% identical to amino acids 20-141 of SEQ ID NO:3.

In some embodiments, the present disclosure provides use of an anti-CCR5 antibody having a heavy chain variable region (VH) that is at least 70% identical to SEQ ID NO:5, at least 75% identical to SEQ ID NO: 5, at least 80% identical to SEQ ID NO: 5, at least 85% identical to SEQ ID NO: 5, or at least 90% identical to SEQ ID NO: 5. In some embodiments the present disclosure provides use of an anti-CCR5 antibody having a heavy chain variable antibody region that is 70%-100% identical to SEQ ID NO: 5, 75%-100% identical to SEQ ID NO: 5, 80%-100% identical to SEQ ID NO: 5, 85%-100% identical to SEQ ID NO: 5, 90%-100% identical to SEQ ID NO: 5, or 91%-100% identical to SEQ ID NO: 5.

In some embodiments, the present disclosure provides use of an anti-CCR5 antibody having a heavy chain variable region (VH) that is at least 70% identical to amino acids 20-141 of SEQ ID NO:5, at least 75% identical to amino acids 20-141 of SEQ ID NO: 5, at least 80% identical to amino acids 20-141 of SEQ ID NO: 5, at least 85% identical to amino acids 20-141 of SEQ ID NO: 5, or at least 90% identical to amino acids 20-141 of SEQ ID NO: 5. In some embodiments the present disclosure provides use of an anti-CCR5 antibody having a heavy chain variable antibody region that is 70%-100% identical to amino acids 20-141 of SEQ ID NO: 5, 75%-100% identical to amino acids 20-141 of SEQ ID NO: 5, 80%-100% identical to amino acids 20-141 of SEQ ID NO: 5, 85%-100% identical to amino acids 20-141 of SEQ ID NO: 5, 90%-100% identical to amino acids 20-141 of SEQ ID NO: 5, or 91%-100% identical to amino acids 20-141 of SEQ ID NO: 5.

In some embodiments, the present disclosure provides use of an anti-CCR5 antibody or an antigen-binding fragment thereof comprising a heavy chain variable region (VH) and a light chain variable region (VL), wherein the VH comprises a heavy chain CDR1 (VH-CDR1) comprising the amino acid sequence of SEQ ID NO:12, a heavy chain CDR2 (VH-CDR2) comprising the amino acid sequence of SEQ ID NO:13, and a heavy chain CDR3 (VH-CDR3) comprising the amino acid sequence of SEQ ID NO:14; and the VL comprises a light chain CDR1 (VL-CDR1) comprising the amino acid sequence of SEQ ID NO:9, a light chain CDR2 (VL-CDR2) comprising the amino acid sequence of SEQ ID NO:10, and a light chain CDR3 (VL-CDR3) comprising the amino acid sequence of SEQ ID NO:11. In some such embodiments, the VH comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:3 or amino acids 20-141 of SEQ ID NO:3, and a VL comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:1 or amino acids 20-131 of SEQ ID NO:1, provided that the amino acid sequences of the VH-CDRs (SEQ ID NOS:12-14) and VL-CDRs (SEQ ID NOS:9-11) are unchanged; or the VH comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:5 or amino acids 20-141 of SEQ ID NO:5, and a VL comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:1 or amino acids 20-131 of SEQ ID NO:1, provided that the amino acid sequences of the VH-CDRs (SEQ ID NOS:12-14) and VL-CDRs (SEQ ID NOS:9-11) are unchanged.

In some embodiments, the present disclosure provides use of an anti-CCR5 antibody or an antigen-binding fragment thereof comprising: (a) a VH comprising an amino acid sequence of SEQ ID NO:3 or amino acids 20-141 of SEQ ID NO:3, and a VL comprising an amino acid sequence of SEQ ID NO:1 or amino acids 20-131 of SEQ ID NO:1; or (b) a VH comprising an amino acid sequence of SEQ ID NO:5 or amino acids 20-141 of SEQ ID NO:5, and a VL comprising an amino acid sequence of SEQ ID NO:1 or amino acids 20-131 of SEQ ID NO:1.

In some embodiments, the present disclosure provides use of an anti-CCR5 antibody comprising a heavy chain (HC) and a light chain (LC). The heavy chain typically comprises a VH and a heavy chain constant region (CH). Depending on the antibody isotype from which it derives, a heavy chain constant region may comprise CH1, CH2, and CH3 domains (IgA, IgD, IgG), or CH1, CH2, CH3, and CH4 domains (IgE, IgM). In some embodiments, the heavy chain constant region comprises a human IgG1, IgG2, IgG3, or IgG4 constant region. In some embodiments, the constant region of the anti-CCR5 antibody is an IgG4 constant region. The light chain typically comprises a VL and a light chain constant region (CL). In some embodiments, a CL comprises a C kappa (“CK”) constant region. In some embodiments, a CL comprises a C lambda (CX) constant region. In some embodiments, an anti-CCR5 antibody of the present disclosure comprises two heavy chains and two light chains, held together covalently by disulfide bridges.

In some embodiments, the present disclosure provides use of an anti-CCR5 antibody comprising a Fc region portion. As used herein, “Fc region portion” refers to the heavy chain constant region segment of the Fc fragment (the “fragment crystallizable” region or Fc region) from an antibody, which can include one or more constant domains, such as CH2, CH3, CH4 or any combination thereof. In some embodiments, an Fc region portion includes the CH2 and CH3 domains of an IgG, IgA, or IgD antibody or any combination thereof, or the CH3 and CH4 domains of an IgM or IgE antibody, and any combination thereof. In some embodiments, a CH2CH3 or a CH3CH4 structure has sub-region domains from the same antibody isotype and are human, such as human IgG1, IgG2, IgG3, IgG4, IgAQ1, IgA2, IgD, IgE, or IgM (e.g., CH2CH3 from human IgG1). By way of background, an Fc region is responsible for the effector functions of an antibody, such as ADCC (antibody-dependent cell-mediated cytotoxicity), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al. Nature 337: 525, 1989). In some embodiments, a Fc region portion in an antibody or antigen-binding fragment of the present disclosure is capable of mediating one or more of these effector functions. In some embodiments, a Fc region portion in an antibody or antigen-binding fragment of the present disclosure has normal effector function, meaning having less than 20%, 15%, 10%, 5%, 1% difference in effector function (e.g., ADCC, CDC, half-life or any combination thereof) as compared to a wild type IgG1 antibody.

In some embodiments, the present disclosure provides use of an anti-CCR5 antibody comprising a Fc region portion having an increase in one or more of these effector functions by way of, for example, one or more amino acid substitutions or deletions in the Fc region portion known in the art. An antibody or antigen-binding fragment having a mutated or variant Fc region portion having increased effector function means that the antibody exhibits an increase of at least 20%, 25%, 30%, 35%, 40%, 45%, 50%, 55%, 60%, 65%, 70%, 75%, 80%, 85%, 90%, 95%, or 99% in FcR binding, ADCC, CDC, or any combination thereof, as compared to an antibody having a wild type Fc region portion. In some embodiments, the mutated or variant Fc region portion exhibits increased binding to FcRn, FcγRI (CD64), FcγRIIA (CD32), FcγRIIIA (CD16a), FcγRIIIIB (CD16b), or any combination thereof. In some embodiments, the Fc region portion in an antibody or antigen-binding fragment of the present disclosure is a variant Fc region portion having increased ADCC, CDC, half-life, or any combination thereof.

Amino acid modifications (e.g., substitutions) to modify (e.g., improve, reduce, or ablate) Fc functionalities include, for example, the T250Q/M428L, M252Y/S254T/T256E, H433K/N434F, M428L/N434S, E233P/L234V/L235A/G236+A327G/A330S/P331S, E333A, S239D/A330L/I332E, P257I/Q311, K326W/E333S, S239D/I332E/G236A, N297Q, K322A, S228P, L235E+E318A/K320A/K322A, L234A/L235A, and L234A/L235A/P329G mutations, which mutations are summarized and annotated in “Engineered Fc Regions”, published by InvivoGen (2011) and available online at www. invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

In some embodiments, the present disclosure provides use of an anti-CCR5 antibody comprising a Fc region portion having a reduction in one or more of these effector functions or lack one or more effector functions by way of, for example, one or more amino acid substitutions or deletions in the Fc region portion known in the art. An antibody or antigen-binding fragment having a mutated or variant Fc region portion having reduced effector function means that the antibody exhibits a decrease of at least 20%, 25%, 30%, 35%, 40%, 45%, 50%, 55%, 60%, 65%, 70%, 75%, 80%, 85%, 90%, 95%, or 99% in FcR binding, ADCC, CDC, or any combination thereof, as compared to an antibody having a wild type Fc region portion. In some embodiments, the mutated or variant Fc region portion exhibits decreased binding to FcRn, FcγRI (CD64), FcγRIIA (CD32), FcγRIIIA (CD16a), FcγRIIIIB (CD16b), or any combination thereof. In some embodiments, the Fc region portion in an antibody or antigen-binding fragment of the present disclosure is a variant Fc region portion having reduced ADCC, CDC, half-life, or any combination thereof. In some embodiments, the Fc region portion is a variant IgG1 Fc region portion comprising a mutation corresponding to amino acid E233P, L234V, L234A, L235A, L235E, AG236, G237A, E318A, K320A, K322A, A327G, P329G, A330S, P331S, or any combination thereof, as numbered according to the EU set forth in Kabat. For example, amino acid substitutions L234A, L235E, G237A introduced into an IgG1 Fc region portion reduces binding to FcγRI, FcγRIIa, and FcγRIII receptors, and A330S and P331S introduced into an IgG1 Fc region portion reduces C1q-mediated complement fixation.

In some embodiments, the present disclosure provides use of an anti-CCR5 antibody comprising a Fc region portion having an increase in one or more of these effector functions by way of, for example, one or more amino acid substitutions or deletions in the Fc region portion known in the art. An antibody or antigen-binding fragment having a mutated or variant Fc region portion having increased effector function means that the antibody exhibits an increase of at least 20%, 25%, 30%, 35%, 40%, 45%, 50%, 55%, 60%, 65%, 70%, 75%, 80%, 85%, 90%, 95%, or 99% in FcR binding, ADCC, CDC, or any combination thereof, as compared to an antibody having a wildtype Fc region portion. In some embodiments, the mutated or variant Fc region portion exhibits increased binding to FcRn, FcγRI (CD64), FcγRIIA (CD32), FcγRIIIA (CD16a), FcγRIIIIB (CD16b), or any combination thereof. In some embodiments, the Fc region portion in an antibody or antigen-binding fragment of the present disclosure is a variant Fc region portion having increased ADCC, CDC, half-life, or any combination thereof.

In some embodiments, the present disclosure provides use of an anti-CCR5 antibody that is glycosylated. IgG subtype antibodies contain a conserved glycosylation site at amino acid N297 in the CH2 domain of the Fc region portion. In some such embodiments, the Fc region portion in an antibody or antigen-binding fragment of the present disclosure comprises a N297 as numbered according to EU set forth in Kabat. In some embodiments, the present disclosure provides use of an anti-CCR5 antibody that comprises a mutation that alters glycosylation at N297 in the Fc region portion, optionally wherein the mutation that alters glycosylation comprises N297A, N297Q, or N297G. In some embodiments, an antibody or antigen-binding fragment thereof comprising a N297A, N297Q, or N297G mutation exhibits reduced Fc interaction with one or more low affinity FcγR(s), reduced CDC, reduced ADCC, or any combination thereof.

In some embodiments, the present disclosure provides use of an anti-CCR5 antibody that comprises a heavy chain (HC) and a light chain (LC), wherein the HC comprises an amino acid sequence that has at least 90%, 91%, 92%, 93%, 94%, 95%, 96%, 97%, 98%, 99%, or 100% identity with the amino acid sequence of SEQ ID NO:7, and the LC comprises an amino acid sequence that has at least 90%, 91%, 92%, 93%, 94%, 95%, 96%, 97%, 98%, or 99% identity with the amino acid sequence of SEQ ID NO:8

In some embodiments, the present disclosure provides use of an anti-CCR5 antibody that comprises a HC comprising an amino acid sequence that has the amino acid sequence of SEQ ID NO:7, and a LC comprising an amino acid sequence that has the amino acid sequence of SEQ ID NO:8.

In some embodiments, the present disclosure provides use of an anti-CCR5 antibody that comprises a Fc region or a fragment thereof, including a CH2 (or a fragment thereof), a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc region of the present disclosure comprises two CH2-CH3 polypeptides that associate to form a dimer.

As used herein, unless otherwise provided, a position of an amino acid residue in the constant region of human IgG1 heavy chain is numbered assuming that the variable region of human IgG1 is composed of 128 amino acid residues according to the Kabat numbering convention. The numbered constant region of human IgG1 heavy chain is then used as a reference for numbering amino acid residues in constant regions of other immunoglobulin heavy chains. A position of an amino acid residue of interest in a constant region of an immunoglobulin heavy chain other than human IgG1 heavy chain is the position of the amino acid residue in human IgG1 heavy chain with which the amino acid residue of interest aligns. Alignments between constant regions of human IgG1 heavy chain and other immunoglobulin heavy chains may be performed using software programs known in the art, such as the Megalign program (DNASTAR Inc.) using the Clustal W method with default parameters. According to the numbering system described herein, for example, although human IgG2 CH2 region may have an amino acid deletion near its amino-terminus compared with other CH2 regions, the position of the “N” located at 296 in human IgG2 CH2 is still considered position 297 because this residue aligns with “N” at position 297 in human IgG1 CH2.

In addition, the present disclosure provides use of an anti-CCR5 antibody that comprises a hinge sequence that is typically situated between the Fab and Fc region (but a lower section of the hinge may include an amino-terminal portion of the Fc region). By way of background, an immunoglobulin hinge acts as a flexible spacer to allow the Fab portion to move freely in space. In contrast to the constant regions, hinges are structurally diverse, varying in both sequence and length between immunoglobulin classes and even among subclasses. For example, a human IgG1 hinge region is freely flexible, which allows the Fab fragments to rotate about their axes of symmetry and move within a sphere centered at the first of two inter-heavy chain disulfide bridges. By comparison, a human IgG2 hinge is relatively short and contains a rigid poly-proline double helix stabilized by four inter-heavy chain disulfide bridges, which restricts the flexibility. A human IgG3 hinge differs from the other subclasses by its unique extended hinge region (about four times as long as the IgG1 hinge), containing 62 amino acids (including 21 prolines and 11 cysteines), forming an inflexible poly-proline double helix and providing greater flexibility because the Fab fragments are relatively far away from the Fc fragment. A human IgG4 hinge is shorter than IgG1 but has the same length as IgG2, and its flexibility is intermediate between that of IgG1 and IgG2. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

In some embodiments, the present disclosure provides use of an anti-CCR5 antibody or antigen binding fragment thereof that is chimeric, humanized, or human. Chimeric and humanized forms of non-human (e.g., murine) antibodies can be intact (full length) chimeric immunoglobulins, immunoglobulin chains or antigen binding fragments thereof (such as Fv, Fab, Fab′, F(ab′)2 or other target-binding subdomains of antibodies), which can contain sequences derived from non-human immunoglobulin. In general, in the humanized antibody or antigen binding fragment thereof most or all of the amino acids outside the CDR regions (e.g., the framework (FR) regions) are replaced with corresponding amino acids derived from human immunoglobulin molecules. In one embodiment of the humanized forms of the antibodies, some, most, or all of the amino acids outside the CDR regions have been replaced with amino acids from human immunoglobulin molecules but where some, most, or all amino acids within one or more CDR regions are unchanged. Small additions, deletions, insertions, substitutions, or modifications of amino acids are permissible as long as they would not abrogate the ability of the antibody to bind a given antigen. A humanized antibody can also comprise at least a portion of a human immunoglobulin constant region (Fc). Suitable human immunoglobulin molecules for use in humanizing a non-human antibody would include IgG1, IgG2, IgG3, IgG4, IgA, and IgM molecules. A “humanized” antibody would retain a similar antigenic specificity as the original antibody, e.g., in the present disclosure, the ability to bind CCR5.

“Human antibodies” can include antibodies having, for example, the amino acid sequence of a human immunoglobulin and include antibodies isolated from human immunoglobulin libraries or from animals transgenic for one or more human immunoglobulins and that typically do not express endogenous immunoglobulins. Human antibodies can be produced using transgenic mice incapable of expressing functional endogenous immunoglobulins, but capable of expressing human immunoglobulin genes. Completely human antibodies that recognize a selected epitope can be generated using guided selection. In this approach, a selected non-human monoclonal antibody, e.g., a mouse antibody, is used to guide the selection of a completely human antibody recognizing the same epitope.

In some embodiments, the present disclosure provides use of an anti-CCR5 antibody or antigen binding fragment thereof that is a part of a multispecific antibody, e.g., a bispecific antibody or a dual variable domain antibody (DVD). Bispecific and DVD antibodies are monoclonal, often human or humanized, antibodies that have binding specificities for at least two different antigens, one of which is CCR5.

In some embodiments, the present disclosure provides use of an anti-CCR5 antibody or antigen binding fragment thereof that are derivatized or otherwise modified. For example, derivatized antibodies can be modified by glycosylation, acetylation, pegylation, phosphorylation, amidation, derivatization by known protecting/blocking groups, proteolytic cleavage, or the like.

In any of the aforementioned embodiments, the anti-CCR5 antibody or antigen binding fragment thereof is conjugated to a small molecule drug to form an antibody drug conjugate.

In some embodiments, the present disclosure provides use of the monoclonal antibody PA14, produced by the hybridoma cell line designated PA14 (ATCC Accession No. HB-12610), or an antigen binding fragment thereof, or an antibody that competes with monoclonal antibody PA-14 in binding to CCR5.

In some embodiments, the present disclosure provides use of leronlimab (PRO140) antibody or antigen binding fragment thereof. Leronlimab (PRO140) is a humanized IgG4 monoclonal antibody that binds to CCR5 described in U.S. Pat. Nos. 7,122,185 and 8,821,877, which are incorporated herein by reference, in their entirety. Leronlimab (PRO 140) is a humanized version of the murine monoclonal antibody, PA14, which was generated against CD4⁺ CCR5⁺ cells. Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999). PRO 140 binds to CCR5 expressed on the surface of a cell, and potently inhibits HIV-1 entry and replication at concentrations that do not affect CCR5 chemokine receptor activity in vitro and in the hu-PBL-SCID mouse model of HIV-1 infection. Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999); Trkola et al., Potent, Broad-Spectrum Inhibition of Human Immunodeficiency Virus Type 1 by the CCR5 Monoclonal Antibody PRO 140, J. VIROL., 75: 579-588 (2001).

Leronlimab does not downregulate CCR5 surface expression or deplete CCR5-expressing cells, but does prevent CCL5-induced calcium mobilization in CCR5+ cells with an IC₅₀ of 45 μg/ml. In some embodiments, a CCR5 binding agent does not downregulate CCR5 surface expression, deplete CCR5-expressing cells, or both. In some embodiments, a CCR5 binding agent inhibits CCL5-induced calcium mobilization of CCR5+ cells with an IC₅₀ of 45 μg/ml. In some embodiments, the CCR5 binding agent is leronlimab.

Leronlimab (PRO 140) binds to CCR5 and blocks viral entry by interfering with the final phase of viral binding to the cell surface prior to fusion of the viral and cell membranes. Leronlimab (PRO 140) has been administered intravenously or subcutaneously to more than 750 healthy and HIV-1 infected individuals in Phase I/II/III studies. The drug has been well tolerated following intravenous administration of single doses of 0.5 to 10 mg/kg or up to 700 mg weekly doses as subcutaneous (SC) injection. Overall, 324 patients have been exposed to leronlimab (PRO 140) 350 mg SC weekly dose with the longest duration of exposure lasting 4 years. Similarly, more than 250 and 150 patients have been exposed to leronlimab (PRO 140) 525 mg and 700 mg SC weekly dose, respectively.

In some embodiments, the present disclosure provides use of an anti-CCR5 antibody that binds to the same epitope as that to which leronlimab binds or competes with leronlimab in binding to CCR5. Leronlimab binds to a discontinuous epitope spanning multiple extracellular domains on CCR5, which include the N-terminus and second extracellular loop (ECL2) of CCR5 (Trkola et al. J. Virol. 75:579-588, incorporated by reference in its entirety). Leronlimab directly blocks binding of HIV Env to the CCR5 co-receptor via a competitive mechanism. Leronlimab binding at least requires amino acid residues D2 in the N-terminus and R168 and Y176 in the ECL2; mutation of amino acids D95 and C101 in the ECL1, and C178 in ECL2 also affect leronlimab binding, e.g., by conformational perturbation (Olson et al. J. Virol. 73:4145-4155, incorporated by reference in its entirety). Targeted loss-of-function mutagenesis and subsequent photo-cross-linking using genetically encoded unnatural amino acids method was also used to map antibody-GPCR complexes and identified residues 174 and 175 at the amino-terminal end of ECL2 as forming the strongest links with leronlimab (Ray-Saha et al., Biochem. 53:1302-13010).

CCR5 amino acid residues that are involved in CCL5 (RANTES) binding include K1, D2, D11, E18, K26 in the N-terminus, D95 in the ECL1, and K171, K191, and R274 in the ECL2 (Navenot et al. J. Mol. Biol. 313:1181-1193, incorporated by reference in its entirety).

Nucleic acids encoding heavy and light chains of the humanized PA140 antibodies have been deposited with the ATCC. Specifically, the plasmids designated pVK-HuPRO140, pVg4-HuPRO140 (mut B+D+I) and pVg4-HuPRO140 HG2, respectively, were deposited pursuant to, and in satisfaction of, the requirements of the Budapest Treaty with the ATCC, Manassas, Va., U.S.A. 20108, on Feb. 22, 2002, under ATCC Accession Nos. PTA 4097, PTA 4099, and PTA 4098, respectively. The American Type Culture Collection (ATCC) is now located at 10801 University Boulevard, Manassas, Va. 20110-2209. The plasmids designated pVK-HuPRO140 and pVg4-HuPRO140 HG2 encode the light chain and heavy chain, respectively, of leronlimab.

The HCDR1-3 and LCDR1-3 amino acid sequences of leronlimab are set forth in SEQ ID NOS:12-14 and 9-11, respectively. The VH and VL sequences of leronlimab are set forth in amino acids 20-141 of SEQ ID NO:3 and amino acids 20-131 of SEQ ID NO:1, respectively. The heavy chain and light chain sequences of leronlimab are set forth in SEQ ID NOS:7 and 8, respectively.

In some embodiments, the present disclosure provides use of an anti-CCR5 antibody comprising: (i) two light chains, each light chain comprising the expression product of the plasmid designated pVK:HuPRO140-VK (ATCC Deposit Designation PTA-4097), and (ii) two heavy chains, each heavy chain comprising the expression product of either the plasmid designated pVg4:HuPRO140 HG2-VH (ATCC Deposit Designation PTA-4098) or the plasmid designated pVg4:HuPRO140 (mut B+D+I)-VH (ATCC Deposit Designation PTA-4099).

In some embodiments, the present disclosure provides use of an anti-CCR5 antibody comprising: (i) two light chains, each light chain comprising the light chain variable (VL) and constant (CL) regions encoded by the plasmid designated pVK:HuPRO140-VK (ATCC Deposit Designation PTA-4097), and (ii) two heavy chains, each heavy chain comprising the heavy chain variable (VH) and constant (CH) regions encoded either by the plasmid designated pVg4:HuPRO140 HG2-VH (ATCC Deposit Designation PTA-4098) or by the plasmid designated pVg4:HuPRO140 (mut B+D+I)-VH (ATCC Deposit Designation PTA-4099).

### Pharmaceutical Compositions

In another aspect, the present disclosure provides use of pharmaceutical compositions comprising leronlimab or fragments thereof and other anti-CCR5 antibodies or fragments thereof described herein for administration to a patient in need thereof. Pharmaceutical compositions can comprise the antibodies or antigen binding fragments described herein and one or more pharmaceutically acceptable carrier, diluent, or excipient, suitable for administration by a selected route. Pharmaceutically acceptable carriers for diagnostic and therapeutic use are well known in the pharmaceutical art, and are described, for example, in Remington's Pharmaceutical Sciences, Mack Publishing Co. (A. R. Gennaro (Ed.), 18^{th} Edition, 1990) and in CRC Handbook of Food, Drug, and Cosmetic Excipients, CRC Press LLC (S. C. Smolinski, ed., 1992). Exemplary pharmaceutically acceptable carriers include any adjuvant, carrier, excipient, glidant, diluent, preservative, dye/colorant, surfactant, wetting agent, dispersing agent, suspending agent, stabilizer, isotonic agent, solvent, emulsifier, or any combination thereof. For example, sterile saline and phosphate buffered saline at physiological pH can be suitable pharmaceutically acceptable carriers. Preservatives, stabilizers, dyes or the like may also be provided in the pharmaceutical composition. In addition, antioxidants and suspending agents may also be used. Pharmaceutical compositions may also contain diluents such as water, buffers, antioxidants such as ascorbic acid, low molecular weight polypeptides (less than about 10 residues), proteins, amino acids, carbohydrates (e.g., glucose, sucrose, dextrins), chelating agents (e.g., EDTA), glutathione, and other stabilizers and excipients. Neutral buffered saline or saline mixed with nonspecific serum albumin are exemplary diluents.

Pharmaceutical compositions comprising an antibody or antigen binding fragment can be manufactured, for example, by lyophilizing the antibody or antigen binding fragment, mixing, dissolving, emulsifying, encapsulating or entrapping the antibody or antigen binding fragment. The pharmaceutical compositions can also include the antibody or antigen binding fragment described herein in a free-base form or pharmaceutically-acceptable salt form.

A pharmaceutical composition may be formulated in the form of a solid, semi-solid or liquid composition. Solid compositions may include powders and tablets. In some embodiments, the pharmaceutical compositions described here are lyophilized or in powder form for re-constitution with a suitable vehicle, e.g., sterile water, before use. In some embodiments, the pharmaceutical compositions described herein is a suspension, solution, or emulsion. The pharmaceutical compositions and formulations can be sterilized. Sterilization can be accomplished by filtration through sterile filtration.

The pharmaceutical compositions described herein can be formulated for oral, topical, transdermal, inhalation, parenteral, sublingual, buccal, rectal, vaginal, and intranasal administration. The term “parenteral”, as used herein, includes subcutaneous, intravenous, intramuscular, intrasternal, and intratumoral injection or infusion techniques. In some embodiments, the pharmaceutical compositions described herein are formulated for administration as an injection, e.g., an intravenous or subcutaneous injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Alternatively, the pharmaceutical compositions described herein can be lyophilized or in powder form for reconstitution with a suitable vehicle, e.g., sterile pyrogen-free water, before use.

The anti-CCR5 antibodies or antigen binding fragments thereof can be formulated for administration in a unit dosage form in association with a pharmaceutically acceptable vehicle. Such vehicles can be inherently nontoxic, and non-therapeutic. A vehicle can be water, saline, Ringer's solution, dextrose solution, and 5% human serum albumin. Non-aqueous vehicles such as fixed oils and ethyl oleate can also be used. The vehicle can contain minor amounts of additives such as substances that enhance isotonicity and chemical stability (e.g., buffers and preservatives).

In some embodiments, an aqueous formulation of a n anti-CCR antibody or antigen binding fragment provided herein, such as for subcutaneous administration, has a pH from 4-5.7. The aqueous formulation may comprise one or more excipients, such as, for example, one or more buffering agents, one or more lyoprotectants, and the like. In some embodiments, the pH of the formulation is from 4.0-6.0, 4.1-5.1, 4.2-5.1, 4.3-5.1, 4.4-5.1, 4.5-5.1, 4-5, 4.1-5, 4.2-5, 4.3-5, 4.4-5, 4.5-5, or about 4.5-5.5, about 5.3, about 5.4, about 5.5, about 5.6, or about 5.7. In some embodiments, the formulation comprises at least one buffer. In various embodiments, the buffer may be selected from histidine, citrate, aspartate, acetate, phosphate, lactate, tromethamine, gluconate, glutamate, tartrate, succinate, malic acid, fumarate, α-ketoglutarate, and combinations thereof. In some embodiments, the buffer is at least one buffer selected from histidine, citrate, aspartate, acetate, and combinations thereof. In some embodiments, the buffer is a combination of histidine and aspartate. In some embodiments, the total concentration of the buffer in the aqueous formulation is 10 mM to 40 mM, such as 15 mM-30 mM, 15 mM-25 mM, or 20 mM.

In some embodiments, the aqueous formulation comprises at least one lyoprotectant. In some such embodiments, the at least one lyoprotectant is selected from sucrose, arginine, glycine, sorbitol, glycerol, trehalose, dextrose, alpha-cyclodextrin, hydroxypropyl beta-cyclodextrin, hydroxypropyl gamma-cyclodextrin, proline, methionine, albumin, mannitol, maltose, dextran, and combinations thereof. In some embodiments, the lyoprotectant is sucrose. In some embodiments, the total concentration of lyoprotectant in the aqueous formulation is 3-12%, such as 5-12%, 6-10%, 5-9%, 7-9%, or 8%.

In some embodiments, the aqueous formulation comprises at least one surfactant. Exemplary surfactants include polysorbate 80, polysorbate 20, poloxamer 88, and combinations thereof. In some embodiments, the aqueous formulation comprises polysorbate 80. In some embodiments, the total concentration of the at least one surfactant is 0.01%-0.1%, such as 0.01%-0.05%, 0.01%-0.08%, or 0.01%-0.06%, 0.01%-0.04%, 0.01%-0.03%, or 0.02%.

In some embodiments, pharmaceutical compositions of the present invention are formulated in a single dose unit or in a form comprising a plurality of dosage units. Methods of preparing such dosage forms are known, or will be apparent, to those skilled in this art; for example, see Remington: The Science and Practice of Pharmacy, 20th Edition (Philadelphia College of Pharmacy and Science, 2000).

In some embodiments, the concentration of the anti-CCR5 antibody or antigen binding fragment in the aqueous formulation is 1 mg/mL-250 mg/mL, such as 10 mg/mL-220 mg/mL, 10 mg/mL-200 mg/mL 10 mg/mL-175 mg/mL, 10 mg/mL-150 mg/mL, 10 mg/mL-100 mg/mL, 20 mg/mL-200 mg/mL, 20 mg/mL-175 mg/mL, 20 mg/mL-150 mg/mL, 20 mg/mL-125 mg/mL, 20 mg/mL-100 mg/mL, 30 mg/mL-200 mg/mL, 30 mg/mL-175 mg/mL, 30 mg/mL-150 mg/mL, 30 mg/mL-125 mg/mL, 30 mg/mL-100 mg/mL, 40 mg/mL-200 mg/mL 40 mg/mL-175 mg/mL, 40 mg/mL-150 mg/mL, 40 mg/mL-125 mg/mL, 40 mg/mL-100 mg/mL, 50 mg/mL-200 mg/mL 50 mg/mL-175 mg/mL, 50 mg/mL-150 mg/mL, 50 mg/mL-125 mg/mL, 50 mg/mL-100 mg/mL, 60 mg/mL-200 mg/mL, 60 mg/mL-175 mg/mL, 60 mg/mL-150 mg/mL, 60 mg/mL-125 mg/mL, 60 mg/mL-100 mg/mL, 70 mg/mL-200 mg/mL, 70 mg/mL-175 mg/mL, 70 mg/mL-150 mg/mL, 70 mg/mL-125 mg/mL, 80 mg/mL-200 mg/mL, 80 mg/mL-175 mg/mL, 80 mg/mL-150 mg/mL, 80 mg/mL-125 mg/mL, 100 mg/mL-200 mg/mL, 125 mg/mL-200 mg/mL, 150 mg/mL-200 mg/mL, or 160 mg/mL-190 mg/mL, 170 mg/mL-180 mg/mL, or 175 mg/mL. In some embodiments, the concentration of the CCR5 binding agent in the aqueous formulation is 100 mg/mL-200 mg/mL. In some embodiments, the concentration of the CCR5 binding agent in the aqueous formulation is 175 mg/mL.

In some embodiments, the anti-CCR5 antibody or antigen binding fragment thereof is formulated in a high protein concentration. High protein concentration formulations containing an exemplary anti-CCR5 antibody are described in U.S. Pat. No. 9,956,165 (incorporated by reference in its entirety).

In some embodiments, the anti-CCR5 antibody or antigen binding fragment is in a formulation comprising concentrated anti-CCR5 antibody or antigen binding fragment thereof in an amount greater than about 100 mg/mL and less than about 200 mg/mL; a tonicifier consisting essentially of a sodium salt and a histidine and glycine buffer present in a combined amount of from about 110 mM to about 120 mM and wherein the buffer is present in an amount of about 10 mM to about 25 mM; and a surfactant, wherein the formulation is hypotonic and has a total salt concentration of less than 100 mM.

In some embodiments, the anti-CCR5 antibody or antigen binding fragment is in a formulation comprising: concentrated anti-CCR5 antibody or antigen binding fragment in an amount greater than about 100 mg/mL and less than about 200 mg/mL; a sodium salt in an amount greater than about 90 mM and less than 100 mM; a histidine and glycine buffer in an amount greater than about 5 mM and less than about 25 mM; a surfactant in an amount greater than about 0.001% w/v and less than about 0.2% w/v; and, optionally, a stabilizing agent or non-salt tonicifier in an amount of about 0.05% w/v to about 1.8% w/v; wherein the formulation has an osmolality of about 250 to about 280 mOsm and has a total salt concentration of less than 100 mM.

In some embodiments, the anti-CCR5 antibody or antigen binding fragment is formulated in a low viscosity, hypotonic formulation, comprising: (a) concentrated anti-CCR5 antibody or antigen binding fragment in an amount greater than about 100 mg/mL and less than about 200 mg/mL; (b) a sodium salt in an amount selected from about 90 mM or about 95 mM; (c) a histidine and glycine buffer in an amount of about 20 mM; (d) a surfactant in an amount of 0.005% to 0.2% w/v; and optionally (e) a stabilizing agent or non-salt tonicifier in an amount sufficient to provide an osmolality of the formulation of about 260-280 mOs/kg; wherein the formulation has a total salt concentration of less than 100 mM.

In some embodiments, the anti-CCR5 antibody or antigen binding fragment is in a low viscosity hypotonic formulation, comprising: (a) concentrated anti-CCR5 antibody or antigen binding fragment in an amount greater than about 100 mg/mL and less than about 200 mg/mL; (b) a salt in an amount selected from about 90 mM or about 95 mM, wherein the salt is selected from sodium chloride, sodium gluconate, or sodium lactate; (c) a histidine and glycine buffer in an amount of about 20 mM; (d) a surfactant in an amount of about 0.005% to about 0.2% w/v, wherein the surfactant is a polysorbate, a poloxamer, or a pluronic; and (e) a stabilizing agent or non-salt tonicifier present in an amount sufficient to provide an osmolality of the formulation of about 230 mOs/kg to about 280 mOs/kg, wherein the stabilizing agent or non-salt tonicifier is selected from a sugar alcohol, a monosaccharide, a disaccharide, or a combination thereof; wherein the formulation has a total salt concentration of less than 100 mM.

In some embodiments, anti-CCR5 antibody or antigen binding fragment is formulated in a composition comprising anti-CCR5 antibody or antigen binding fragment in an amount greater than about 100 mg/mL and less than about 200 mg/mL, a tonicifier comprising a sodium salt present in a concentration of greater than about 90 mM and a histidine and glycine buffer present in a combined amount of from 110 mM to 120 mM and a surfactant present in an amount of from about 0.001% to about 0.2% w/v, wherein the composition has an osmolality of about 230 to about 290 mOs/kg and a total salt concentration of less than 100 mM.

In some embodiments, anti-CCR5 antibody or antigen binding fragment is provided as an article of manufacture comprising a container and a formulation comprising anti-CCR5 antibody or antigen binding fragment in a concentration of greater than 100 mg/mL and less than 200 mg/mL, a tonicifier of a sodium salt present in a concentration of greater than about 90 mM and a histidine and glycine buffer present in a combined amount of from about 110 mM to about 120 mM and the formulation has a total salt concentration of less than 100 mM, a surfactant in an amount of from about 0.005% to about 0.2%, and instructions for use.

In some embodiments, anti-CCR5 antibody or antigen binding fragment is administered in a dose of 700 mg of anti-CCR5 antibody or antigen binding fragment (175 mg/mL) delivered as two injections of 2 mL each and administered subcutaneously on opposite sides of the abdomen. Each vial of the anti-CCR5 antibody or antigen binding fragment product may contain ~1.4 mL antibody at a concentration of 175 mg/mL.

In any of the aforementioned pharmaceutical compositions, the anti-CCR5 antibody may be leronlimab.

### Methods of Use

The CCR5 receptor is a C-C chemokine G-coupled protein receptor expressed on lymphocytes (e.g., NK cells, B cells), monocytes, monocytes, dendritic cells, a subset of T cells, etc. The extracellular portions represent potential targets for antibodies targeting CCR5, and comprise an amino-terminal domain (Nt) and three extracellular loops (ECL1, ECL2, and ECL3). The extracellular portions of CCR5 comprise just 90 amino acids distributed over four domains. The largest of these domains are at the Nt and ECL2 at approximately 30 amino acids each (Olson et al., Curr. Opin. HIV AIDS, March, 4(2): 104-111 (2009)).

The CCR5 receptor binds to a chemokine known as CCL5 (C-C chemokine ligand 5), which is an inflammatory chemokine that plays an important role in immunologic mechanisms such as controlling cell recruitment and activation in basal and inflammatory circumstances. CCL5 acts as a key regulator of CCR5+ cell (e.g., monocyte and T cell) migration to inflammatory sites, directing migration of monocytes and T cells to damaged or infected sites. CCR5 also plays a crucial role in differentiation and activation of CD8+ T cells. Many biologic effects of chemokines are mediated by their interaction with chemokine receptors on cell surfaces. The most relevant known receptor for CCL5 is the CCR5 receptor; however, CCR1 and CCR3 are also known CCL5 receptors and CCR4 and CD44 are auxiliary receptors. Tamamis et al., Elucidating a Key Anti-HIV-1 and Cancer-Associated Axis: The Structure of CCL5 (Rantes) in Complex with CCR5, SCIENTIFIC REPORTS, 4: 5447 (2014).

The formation of the CCL5 ligand and CCR5 receptor complex causes a conformational change in the receptor that activates the subunits of the G-protein, inducing signaling and leading to changed levels of cyclic AMP (cAMP), inositol triphosphate, intracellular calcium and tyrosine kinase activation. These signaling events cause cell polarization and translocation of the transcription factor NF-kB, which results in the increase of phagocytic ability, cell survival, and transcription of proinflammatory genes.

Treatment with a CCR5 binding agent, such as leronlimab, may affect key anti-inflammatory mediators or other biomarkers, as disclosed herein, in NASH and NAFLD patients. In particular, CCR5L (RANTES) production may increase as a direct result of CCR5 stabilization on the surface of immune cells by a CCR5 binding agent, such as leronlimab. CCR5/RANTES binding and signaling is an important mediator in the activation of CD8+ T cells.

Several reports have shown that CCR5 and CCR2 are capable of forming heterodimers and heterodimerization preferentially alters the signaling outcome from chemotaxis to cell adhesion. Treatment with a CCR5 binding agent, such as leronlimab, significantly increases surface stability of CCR5, which increase the chances of its heterodomerization with CCR2 on cells expressing both receptors. Although heterodimerization of CCR5/CCR2 does not preclude transcellular migration, a CCR5 binding agent, such as leronlimab-bound CCR5 prevents transcellular migration, which is likely a key mechanism by which a CCR5 binding agent, such as leronlimab, restores vessel integrity and restitution of the immune function.

Nonalcoholic fatty liver disease (NAFLD) is a set of conditions in which fat deposits are formed in the liver. NAFLD includes nonalcoholic fatty liver (NAFL) and nonalcoholic steatohepatitis (NASH). NAFL is characterized by the buildup of fat without substantial accompanying inflammation or liver damage. NAFL may cause pain in the patient, but rarely results in clinically relevant liver damage. NASH is characterized by the presence of both fatty deposits and inflammation in the liver. This inflammation can lead to fibrosis in the liver and other complications, such as cirrhosis or liver cancer. CCR5 and CCR2 have been strongly implicated in the development of NASH and related fibrosis. (See e.g. Lefere S, Devisscher L, Tacke F. Targeting CCR2/5 in the treatment of nonalcoholic steatohepatitis (NASH) and fibrosis: opportunities and challenges. Expert Opin Investig Drugs. 2020 February; 29(2):89-92. doi: 10.1080/13543784.2020.1718106. Epub 2020 January 20. PMID: 31952447.) Accordingly CCR5 and CCR2 dysregulation may play a significant role in the development of these conditions, such that treatment with a CCR5 binding agent may be beneficial. Details of methods and uses of the CCR5-binding agent leronlimab to treat NASH and related symptoms or resulting further disorders, such as fibrosis and other scarring, are provided in the Examples. Details of these methods and uses, including doses and dosing regiments and target patient populations may be combined with any of the present disclosure regarding CCR5 and CCR2 regulation and the administration of CCR5 binding agents. In particular, a CCR5 binding agent, such as leronlimab, may reduce inflammation in the liver. Leronlimab may be administered in any dose on any schedule, but, in a particular embodiment, may be administered in a dose of 350 mg or 700 mg once weekly by intravenous injection. Inflammation in the liver may be measured by circulating cytokine levels in the patient's blood or by biopsy. Reduction in inflammation, fatty deposits, fibrosis, scarring, or cirrhosis may also be measured by MRI or other medical imaging methods, liver function tests, or by any methods used clinically to assess these symptoms. MRI-based assessments may include (Proton Density Fat Fraction) PDFF and iron-corrected T1 mapping (cT1) assessments. Therapeutic effect may be measured by decrease from pre-treatment baseline in hepatic fat fraction, assessed MRI-PDFF. This change may be assessed six, twelve, fourteen, twenty or twenty four weeks after the first dose of CCR5 binding agent. Therapeutics effect may also be measured by a decrease from pre-treatment baseline in fibro-inflammatory activity in the liver as assessed by cT1 This change may also be assessed six, twelve, fourteen, twenty or twenty four weeks after the first dose of CCR5 binding agent.

The disclosure further provides methods of treating NASH, NAFLD, and associated symptoms and disorders by administering leronlimab to a patient with NASH or NAFLD, or at increased risk of developing NASH or NAFLD.

Patients with NAFL may be at an increased risk of developing NASH. Patients with NAFLD who have increased levels of biomarkers associated with inflammation may be at an increased risk of developing NASH. Patients with abnormal PDFF and/or CT1 MRI results may be at an increased risk of developing NASH.

CCR5 haplotype pairs (genotypes) based on the CCR5 promoter polymorphism at position −2459 are known. The single nucleotide polymorphism (SNP) at −2135 is in 100% linkage with the SNP at the position −2459, such that −2459G is always linked with −2135T and −2459A is always linked with −2135C. A CCR5 haplotype not associated with increased CCR5 cell surface expression may include type A/A for the SNP 2459. HHE and HHG haplotypes are often associated with increased CCR5 cell surface expression. The effects of CCR5 haplotypes in HIV is described in Catano G, Chykarenko Z A, Mangano A, et al. Concordance of CCR5 genotypes that influence cell-mediated immunity and HIV-1 disease progression rates. J Infect Dis. 2011; 203(2):263-272. doi:10.1093/infdis/jiq023, which is incorporated by reference herein.

Patients with a CCR5 haplotype associated with overexpression of CCR5 and high surface levels of CCR5, particularly the HHE and HHG haplotypes, may be at an increased risk of developing NASH.

Patients may include a mammal, such as human or non-human primates. In certain embodiments, the patient is human. The patient can be male or female and can be any suitable age, including infant, juvenile, adolescent, adult, and geriatric, although the presence of NASH is typically more likely in older patients.

The patient may be administered leronlimab once or for a set period of time, such as 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 6 months, 9 months, 1 year, 2 years, until the propensity to develop NASH has resolved, until at least one symptom of NASH has resolved, or until at least one MRI imaging indicator or biomarker of NASH has been returned to a normal level or a level not associated with the presence of NASH. In some embodiments, leronlimab may be administered until serum RANTES levels are sufficiently low to have no clinical inflammatory effect on NASH. Administration may be recommended in an MRI or biomarker level moves in a direction indicating redevelopment or worsening of NASH, or when RANTES levels are no longer sufficiently low to have no clinical inflammatory effect on NASH.

For NASH patients, optimized RANTES levels reflect neutral binding of CCR5 by leronlimab. This RANTES level is very different than what is desirable in HIV patients, for whom as much CCR5 binding by leronlimab as possible is desirable to prevent HIV binding and infection of cells. In NASH, if too much leronlimab is provided, resulting in over binding of CCR5, the immune response is increased. A lower dose of leronlimab results in neutral CCR5 binding, which causes an alteration in immune function, but not an increase in immune response.

An appropriate dose, suitable duration, and frequency of administration of the leronlimab will be determined by such factors as the condition of the patient, size, weight, body surface area, age, sex, type and severity of the disease, particular therapy to be administered, particular form of the active ingredient, time and the method of administration, and other drugs being administered concurrently, which can readily be determined by a person skilled in the art.

Dosages can range from 0.1 to 100,000 g/kg. Based upon the composition, the dose can be delivered continuously, such as by continuous pump, or at periodic intervals, e.g., on one or more separate occasions. Desired time intervals of multiple doses of a particular composition can be determined without undue experimentation by one skilled in the art.

In some embodiments, the leronlimab is administered to the patient a plurality of times and each administration delivers from 0.01 mg per kg body weight to 50 mg per kg body weight of the antibody or binding fragment thereof to the patient. In another embodiment, each administration delivers from 0.05 mg per kg body weight to 25 mg per kg body weight of the leronlimab to the patient. In a further embodiment, each administration delivers from 0.1 mg per kg body weight to 10 mg per kg body weight of the leronlimab to the patient. In a still further embodiment, each administration delivers from 0.5 mg per kg body weight to 5 mg per kg body weight of the leronlimab to the patient. In another embodiment, each administration delivers from 1 mg per kg body weight to 3 mg per kg body weight of the leronlimab to the patient. In another embodiment, each administration delivers about 2 mg per kg body weight of the leronlimab) to the patient.

The leronlimab may be administered once, twice, or a plurality of times.

In one embodiment, the leronlimab is administered a plurality of times, and a first administration is separated from the subsequent administration by an interval of less than one week. In another embodiment, the first administration is separated from the subsequent administration by an interval of at least one week. In a further embodiment, the first administration is separated from the subsequent administration by an interval of one week. In another embodiment, the first administration is separated from the subsequent administration by an interval of two to four weeks. In another embodiment, the first administration is separated from the subsequent administration by an interval of two weeks. In a further embodiment, the first administration is separated from the subsequent administration by an interval of four weeks. In yet another embodiment, leronlimab is administered a plurality of times, and a first administration is separated from the subsequent administration by an interval of at least one month. In yet another embodiment, the leronlimab is administered once a week for two weeks. In yet another embodiment, the leronlimab is administered once a week for four weeks. In yet another embodiment, the leronlimab is administered once per week as long as needed.

In a further embodiment, the leronlimab is administered to the patient via intravenous infusion. In another embodiment, the leronlimab is administered to the patient via subcutaneous injection. In another embodiment, the leronlimab is administered to the patient via intramuscular injection.

In some embodiments, the leronlimab is administered at a once weekly dose of 350 mg to 1400 mg, or about 525 mg or about 700 mg or about 1050 mg. In some embodiments, the leronlimab is administered at a twice weekly dose of 350 mg to 1400 mg, or about 525 mg or about 700 mg or about 1050 mg. In some embodiments, the leronlimab is administered at a dose of about 700 mg, once weekly.

In some embodiments, the leronlimab is administered in a manner and dose similar to that used to treat HIV infection.

Leronlimab (PRO 140) is currently approved or under development for the indications of HIV, Graft versus host disease (GVHD), metastatic triple negative breast cancer (mTNBC), metastatic colorectal cancer (mCRC), and acute or long-COVID. The safety profile of leronlimab (PRO 140) has been extensively evaluated in clinical trials. Leronlimab (PRO 140) has been administered intravenously or subcutaneously to more than 750 healthy and HIV-1 infected individuals in Phase I/II/III studies. The drug has been well tolerated following intravenous administration of single doses of 0.5 to 10 mg/kg or up to 700 mg weekly doses as subcutaneous (SC) injection. Overall, 324 patients have been exposed to leronlimab (PRO 140) 350 mg SC weekly dose with the longest duration of exposure lasting 4 years. Similarly, more than 250 and 150 patients have been exposed to leronlimab (PRO 140) 525 mg and 700 mg SC weekly dose, respectively.

In some embodiments, leronlimab is administered in a dose of 700 mg (175 mg/mL) delivered as two subcutaneous injections of 2 mL each of which may be on opposite sides of the abdomen. In some embodiments, leronlimab is administered in a dose of 525 mg or 350 mg delivered as one or two subcutaneous injections of 2 mL, which may be on opposite sides if two injection are used.

In some embodiments, the dose of leronlimab is determined by the patient's CCR5 haplotype, with patients having a CCR5 haplotype associated with increased cell surface expression of CCR5, such as a HHE or HHG haplotype, receiving a higher dose of leronlimab or more frequent administration as compared to patients without such a haplotype. For example, patients with a CCR5 haplotype associated with higher cell surface levels of CCR5 may be administered a dose of 525 mg or 700 mg of leronlimab weekly, or a dose of 350 mg or 535 mg twice a week.

In some embodiments, the appropriate leronlimab dose is determined by measuring baseline levels of an MRI indicator or biomarker, administering a low dose of leronlimab, such as 350 mg per week, for a short duration of time, such as one week, two weeks, three weeks, on month, or two months, then measuring the MRI indicator or biomarker to determine any changes. If a change does not exceed a pre-set level or if the overall level of the MRI indicator or biomarker does not meet a set parameter (such as a serum RANTES level of approximately zero, which may be less than 0.01 ng/mL), then the dose of leronlimab may be increased, for example to 525 mg or 700 mg per week, or the frequency of administration may be increased, for example to twice per week, for a short duration of time, then the MRI indicator or biomarker may be measured again and compared to pre-set changes or overall levels. If a set parameter is not met, then the dose or frequency may be further increased, for example to a dose of 700 mg once per week. Once an effective dosing regimen has been determined, the patient may be continued on that dosing regimen, or a lower dosing regimen may be periodically tested, with measurement of effects on the MRI indicator or biomarker, to determine if the lower dosing regimen is effective at that time. Some patients may be able to maintain clinical benefits at a lower leronlimab dose or discontinue leronlimab entirely as inflammation decreases.

Specific biomarkers for NASH may include (i) chemokines, (ii) CCLs, specifically CCL 2, 3, 5, 11, and 18, (iii) interleukins, (iv) adhesion markers such as VCAM, (v) apoptosis and necrosis markers, specifically mitochondrial CK18 m30 and 65 apoptosis and necrosis. Biomarker testing may test for multiple members of one of these groups, or one or more members of more than one, two or more, three or more, four or more, or all of these groups.

### EXAMPLES

### Example 1: Pre-Clinical Studies of Leronlimab for the Treatment of NASH

The potential for leronlimab in the treatment of NASH was demonstrated in a pre-clinical model of fatty liver disease.

Immunodeficient NOD scid IL-2 receptor gamma knockout mice (NSG) were fed a high fat NASH-inducing diet, transplanted with human stem cells to repopulate the deficient immune system, and treated with leronlimab or an IgG control. 16 male were first humanized by intravenous inoculation with normal human umbilical cord blood cells. After 5 weeks on normal mouse chow, mice were successfully humanized, demonstrating >25% human CD45 cells in peripheral blood. Mice were switched to a high fat (52%), high cholesterol (1.25%) diet. (FPC diet: fructose, palmitate, cholesterol, trans-fat; Envigo-Teklad TD.160785). Leronlimab and control antibody (normal human IgG, Sigma) were delivered via intraperitoneal (i.p.) administration at a dose of 2 mg i.p. twice weekly, n=8 mice/group. Mice were euthanized at 16 weeks after initiation of the high fat, high cholesterol diet.

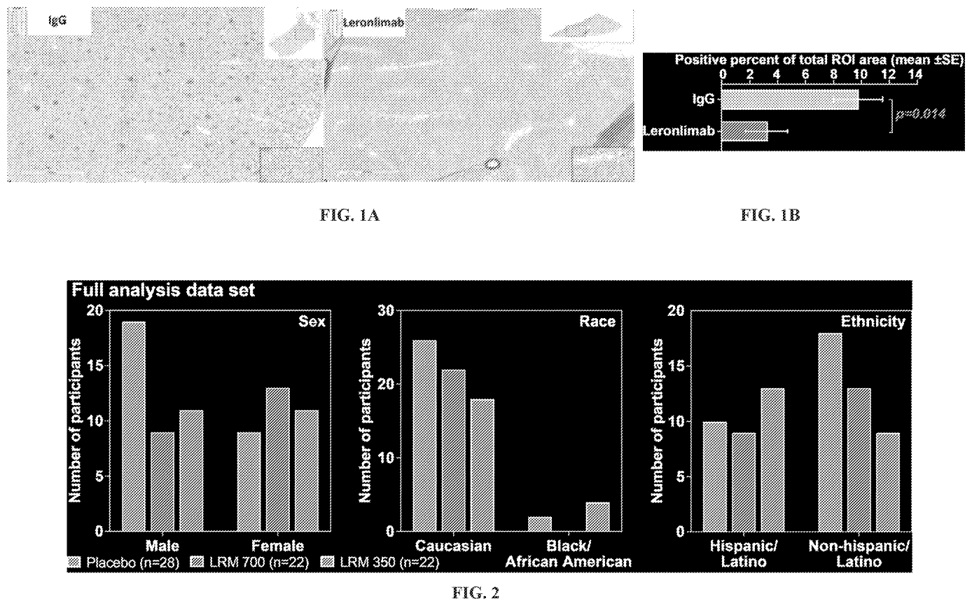

Liver sections were analyzed for indicators of NAFLD. Representative 20× microscopy images from leronlimab and generic IgG-treated mice are presented in FIG. 1A. Leronlimab-treated mice exhibited a markedly reduced presence of fat in the liver, as indicated by Oil Red O stain. Regions of interest were identified in micrographs and digitized using an Aperio AT2 slide scanner (Leica Biosystems), then analyzed using QuPath v0.2.01 imaging software. Combined results from all mice in the study are presented in FIG. 1B. The graph shows mean Oil Red O positive pixels, relative to the entire region of interest (ROI) and standard error (SE) calculated for both treatment groups. Percent positivity in IgG treated group was 9.751±1.789. Percent positivity in Leronlimab treated group was 3.207±1.515. Student's t test p=0.014. Steatosis was numerically scored following a semi-quantitative pathological standard.

The results show that leronlimab inhibited fatty liver development, a key characteristic of early-stage NASH.

### Example 2: Clinical Studies of Leronlimab for the Treatment of NASH

### Study Objectives

A clinical study was conducted to evaluation the potential for leronlimab in the treatment of NASH. The trial, referenced as CDI-NASH-01, establishes that leronlimab may be used to treat NASH and symptoms and further disorders resulting from NASH, as indicated below.

### Study Design

CDI-NASH-01 was designed as a multi-center Phase 2a trial and was subsequently converted into an exploratory study to evaluate dose, efficacy, and safety of leronlimab at doses of 700 mg and 350 mg for the treatment of NASH. Biomarkers were also measured to help design future trials and understand the potential mechanisms of action of leronlimab.

The primary objective of the Part 1 study was to assess the efficacy of 700 mg leronlimab (n=22) in improving NASH tests in adult patients diagnosed with NASH/NAFLD compared to placebo (n=28). Part 2 was subsequently added to assess the efficacy of 350 mg leronlimab in improving NASH/NAFLD tests in adult patients diagnosed with NASH (n=22).

The secondary objective of this study was to assess the safety and tolerability of leronlimab in adult patients diagnosed with NASH compared to placebo.

An overview of the study and related timeline are as follows:

### Part 1:

Week −4 to Week 0—Screening
Treatment Allocation (Lleronlimab vs. placebo) occurred at the beginning of Week 0
Week 0 to Week 1—Double-blind, placebo-controlled study (randomized 1:1)
Week 1 to Week 14—Treatment given weekly (+/−1 days relative to last dose)
Week 13 to Week 14—End of treatment visit
Week 14 to Week 18—Follow-up

### Part 2

Week −4 to Week 0—Screening
Treatment Allocation (Leronlimab) occurred at the beginning of Week 0
Week 0 to Week 14—Single-arm Open-label treatment given weekly (+/−1 days relative to last dose)
Week 13 to Week 14—End of treatment visit
Week 14 to Week 18—Follow-up

In both Part 1 and Part 2, leronlimab was given subcutaneously. In both Part 1 and Part 2, Follow-up was 28 (+/−) 3 days after End of Treatment (EOT) or Early Termination (ET) visit.

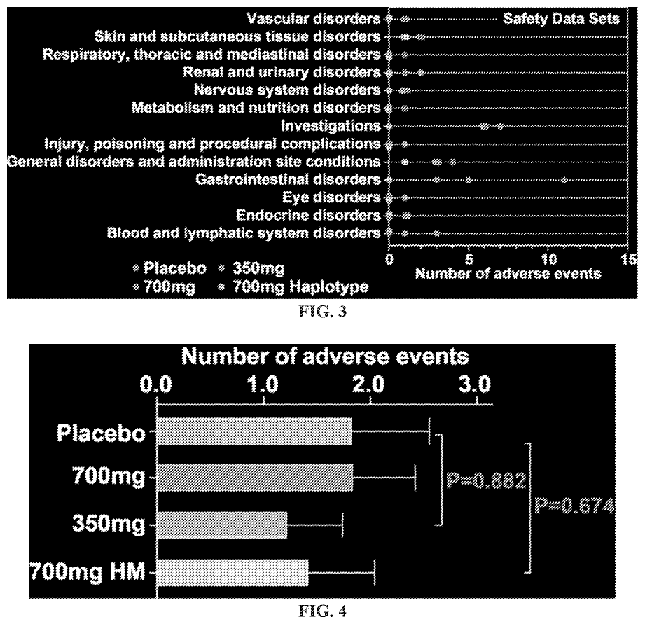

Eligible patients included adults aged between 18 to 75 years (inclusive), with evidence of phenotype nonalcoholic steatohepatitis (NASH). Patients had a Body Mass Index (BMI)≥28 kg/m² and were required to demonstrate the presence of hepatic fat fraction, as defined by ≥8% on MRI-(Proton Density Fat Fraction) PDFF and iron-corrected T1 mapping (cT1)≥800 milliseconds (msec) at screening. A stable body weight (±5%) was required during the 6 months prior to screening. Exclusion criteria include but were not limited to HIV, autoimmune hepatitis, excess alcohol use, viral hepatitis, and prior or pending liver transplantation. No patients in the 350 mg study were concomitantly treated with semaglutide, while 18% of patients in the placebo group were concomitantly treated with semaglutide. Patient demographics are summarized in FIG. 2.

Analyses were conducted on the full analysis set (n=72) of whom 44% were of Hispanic or Latino ethnicity and 58% with baseline moderate to severe fibro-inflammation (cT1≥875 ms).

### Study Treatment

Subjects who met all eligibility criteria, as per data gathered from the screening period, qualified for enrollment. All subjects who failed to meet eligibility criteria were considered screen failures and exited the study without further evaluation. The study consisted of two parts:

Part 1 of the study, eligible subjects randomized to 1:1 to one of the two study arms to receive leronlimab 700 mg (Group A), or placebo (Group B) given once per week (±1 day) at the study site for up to 13 weeks during the treatment period (with up to 60 participants).

Part 2 of the study, eligible subjects enrolled to receive leronlimab 350 mg open label given one per week (1 day) at the study site for up to 13 weeks during the treatment period (with up to 28 participants). There were 21 patients in cT1 analysis and 22 for PDFF analysis. One patient obtained PDFF at End of Treatment but MRI for cT1 despite repeat was not able to quality (by 3 cuts) so no results for cT1 were obtained on that patient. This patient did have severe cT1 at baseline thus the numbers for all patients administered 350 mg leronlimab, patients with moderate NASH administered 350 mL leronlimab, and patients with severe NASH administered 350 mL leronlimab were all reduced from that seen in PDFF.

The primary efficacy objective of the study was the change from baseline in hepatic fat fraction, assessed by magnetic resonance imaging-derived proton density fat fraction (MRI-PDFF) at week 14.

The secondary efficacy objective was the change from baseline in fibro-inflammatory activity in the liver as assessed by cT1 (corrected T1) at week 14. cT1 is obtained by multiparametric magnetic resonance imaging of the liver and is a quantitative metric for assessing a composite of liver inflammation and fibrosis, expressed in milliseconds (msec). Additional objectives were change from baseline in LFT, chemokine and cytokine levels and key biomarkers of inflammation to week 14. In particular, change in serum cK18 and K18 by M30-M65 ELISA was assessed at week 14.

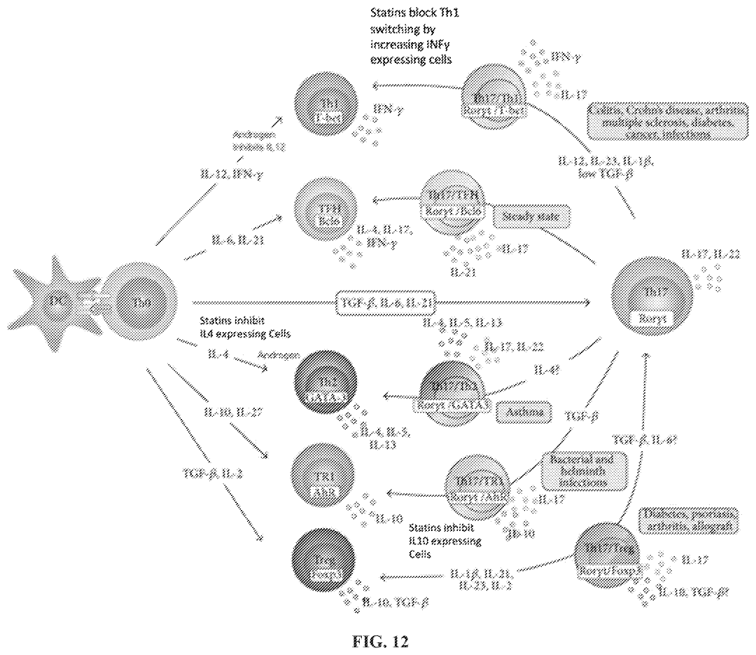

Over transcription of CCR5 due to the HHG allele is associated with increased surface CCR5 on T cells but not monocytes, and is associated with reduced T cell immunity, reduced T cell proliferation, reduced T cell differentiation. Patients with over transcription of CCR5 may have delayed hypersensitivity response and impaired Th1. Possible pathways associated with these effects are provided in FIG. 12. The effects of CCR5 haplotypes in HIV is described in Catano G, Chykarenko Z A, Mangano A, et al. Concordance of CCR5 genotypes that influence cell-mediated immunity and HIV-1 disease progression rates. J Infect Dis. 2011; 203(2):263-272. doi:10.1093/infdis/jiq023, which is incorporated by reference herein.

CCR5 haplotype was investigated for 5 patients. Haplotypes with very high transcription and thus high cell surface expression of CCR5 include HHE/HHE and HHG1/HHG1. High transcription alleles when combined with a null allele delta 32 or HHG2 will default to the heterozygous allele such that HHE/HHG2 and HHG1/HHG2 would also be high transcribers for CCR5. Other combinations including HHE/HHG1 were also included in the patient population exhibiting high transcription.

### Results

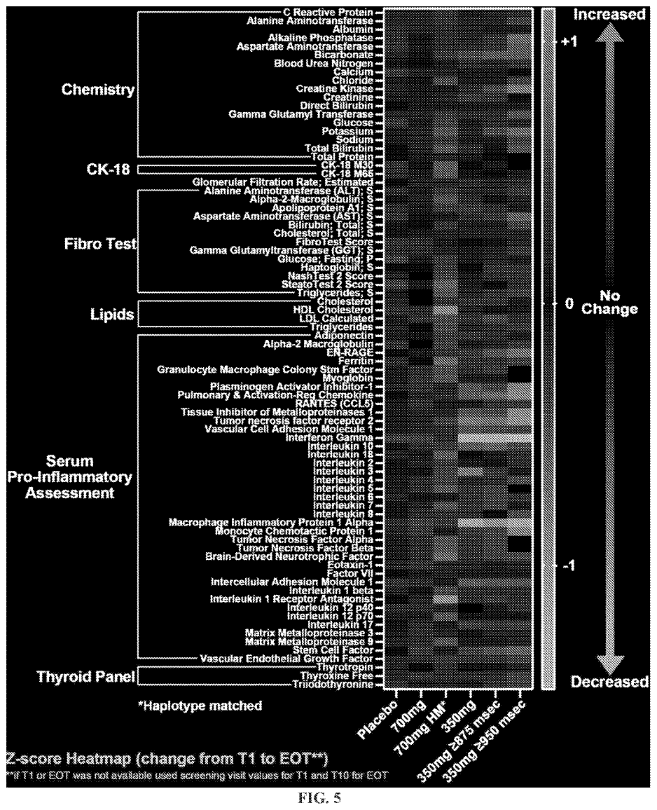

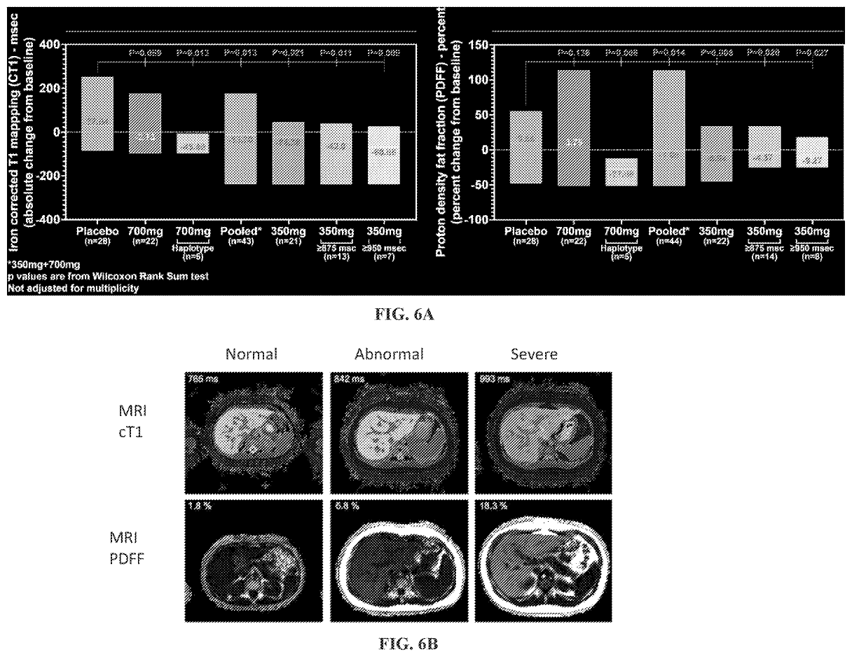

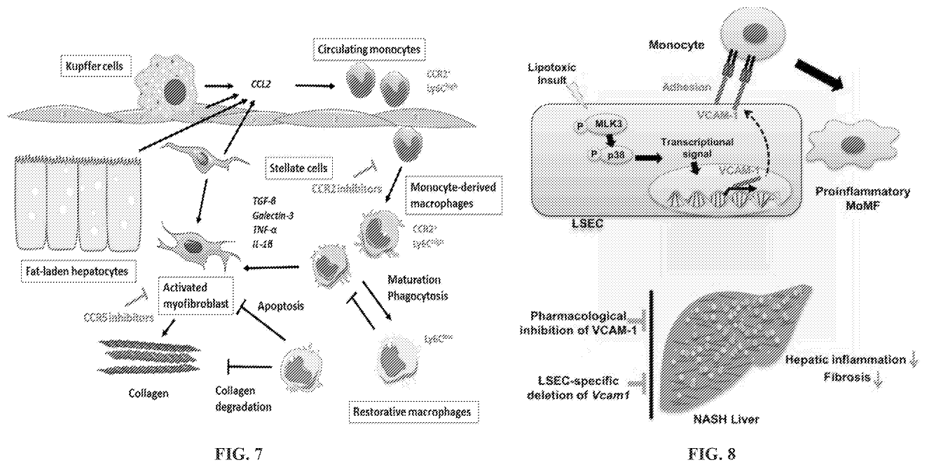

Treatment with leronlimab was generally well tolerated in both Part 1 and Part 2 of the study. (See FIG. 3 and FIG. 4.) There was no grade 3 or higher drug related treatment emergent adverse event. Injection site reaction and mild diarrhea occurred more frequently with leronlimab than placebo but were not associated with discontinuation.
Part 1: Leronlimab 700 mg did not reduce mean change in proton density fat fraction (PDFF) and cT1 from baseline to week 14 as compared to the placebo.
Part 2: Leronlimab 350 mg significantly reduced mean change in PDFF and cT1 from baseline to week 14 as compared to the placebo.

Part 1 and 2 pooled: Pooled 350 mg and 700 mg group also had significant reductions in PDRR and cT1 as compared to the placebo. Although small sample size (n=5) CCR5 haplotype analysis is suggestive that specific haplotypes may be better suited from the 700 mg dose of leronlimab.

Analysis grouped certain patients by other differentiating factors such as i) the presence of moderate NASH, as defined by a baseline cT1≥875 ms, or severe NASH, as defined by a baseline cT1≥950 ms; and ii) CCR5 haplotype.

### MRI Analysis:

MRI PDFF and cT1 results are provided in FIG. 6A. Representative MRI images are provided in FIG. 6B.

Mean percent change from baseline PDFF was significantly reduced in the 350 mg group vs placebo (−5.94% vs+9.85%, p=0.008) but not in the 700 mg group (+3.75% vs +9.85%, p=0.135). (See Table 1 and Table 2.) Mean change cT1 was significantly reduced in the 350 mg group vs placebo (−24.38 ms vs+27.64 ms, p=0.021) but not in the 700 mg group (−2.73 ms vs+27.64 ms, p=0.059). Significant reductions were seen in the 350 mg subgroup with baseline cT1≥875 ms in both PDFF and cT1 vs placebo (−4.37% vs+9.85%, p=0.020 and −42.00 ms vs+27.64 ms, p=0.011) respectively. In subjects with cT1≥950 ms at baseline, PDFF and cT1 were significantly reduced with 350 mg vs placebo (−9.39% vs +9.85%, p=0.027 and −68.85 ms vs+27.64, p=0.009) respectively. (See Table 1) In post hoc analyses, mean percent PDFF and mean cT1 were significantly reduced in the pooled 350+700 mg group compared to placebo (−1.09% vs+9.85%, p=0.014 and −13.30 ms vs+27.64 ms, p=0.013) and in the 700 mg group with genetic haplotypes known to over produce CCR5 compared to placebo (−27.9% vs+9.85%, p=0.006 and −45.4 ms vs+27.64 ms, p=0.013).

Overall, where fibroinflammation was more pronounced, patients exhibited more pronounced reductions in cT1, with a decrease of up to −69 ms in patients with severe NASH.

### Serum Analysis:

A heatmap of changes in blood chemistry, including cytokines and other markers, in study patients is provided in FIG. 5. Additional data in which patients were separately analyzed based on cT1 levels is presented in Table 1.

Mean change in baseline to week 14 for M65 ELISA (cK18 and K18) decreased in the 350 mg group (340.55 to 332.4 U/L; −8.18) while increased in placebo (301.96 to 411.64 U/L; +109.78).

Reductions in common liver function biomarkers ALT, AST and Alkaline Phosphatase were observed in the 350 mg group and cT1 subgroups vs placebo. Specifically, patients with baseline elevated ALT (n=8) exhibited reductions in ALT when administered 350 mg leronlimab as compared to a placebo (mean 83.7 to 54.3; −29.3 U/L vs. 87.4 to 83.2; −4.2 U/L) with corresponding reductions in PDFF (−18% vs. 6%) and cT1 (−69 ms vs. 5 ms).

Reductions in the chemotactic proteins CCL2, CCL3, CCL11, and CCL18 were also observed in patients administered 350 mg leronlimab, but balanced or increased levels of these biomarkers were observed in patients administered 700 mg leronlimab (although different results may be expected in patients with haplotypes corresponding with increased surface CCR5 expression).

VCAM and EN RAGE levels were also reduced by the administration of 350 mg leronlimab. A potential mechanism of action suggested by these reductions is illustrated in FIG. 8.

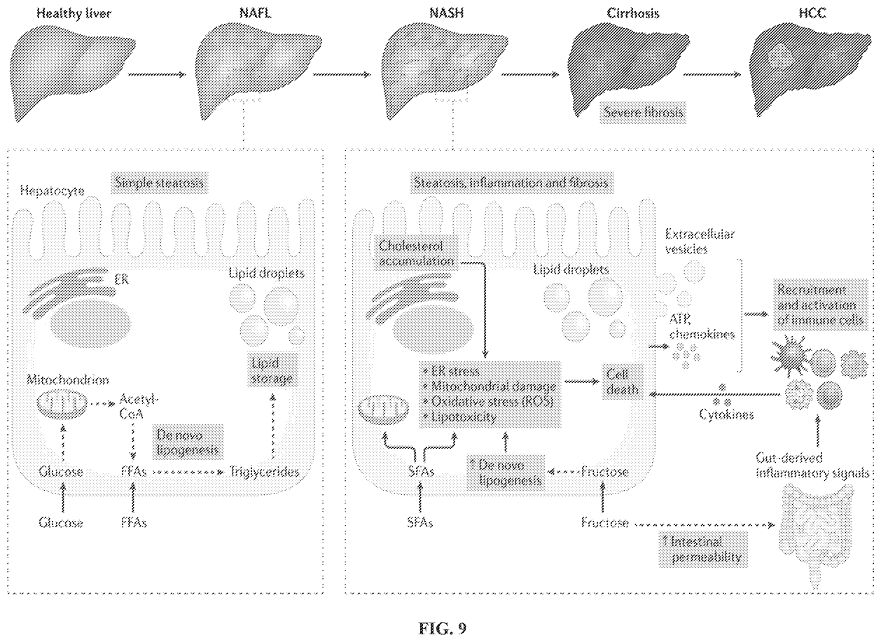

IL-L Beta, IL-1 IRA, IL-6, IL-8 and TNF Receptor 2 levels were also reduced in patients administered 350 mg leronlimab. A potential mechanism of action suggested by these reductions is illustrated in FIG. 9.

Cardiovascular biomarkers were also positively affected by leronlimab. Favorable increases in Apolipoprotein A1S and HDL levels were also observed in patients administered 350 mg leronlimab, as was a reduced neutrophil to lymphocyte ration, reductions in TIMP-1 (which may correlate with reductions in fibrogenic pathway activation), and reductions in EN RAGE and VCAM (which likely have systemic implications, such as in endothelial inflammation). A potential mechanism of action of leronlimab on cardiovascular effects suggested by these markers is provided in FIG. 11.

TABLE 1
350 mg Leronlimab Based on cT1 levels vs. Placebo

| | Placebo | 350 mg leronlimab | 350 mg cT1 ≥ 875 | 350 mg cT1 ≥ 950 |
|---|---|---|---|---|
| Biomarker (Baseline to Week 14) | (n = 28) | (n = 22) | (n = 14) | (n = 8) |
| MRI PDFF % | 9.85 | −5.94** | −4.38* | −9.27* |
| MRI cT1 | 27.64 | −24.38** | −42.00** | −68.86** |
| Alkaline Phosphatase IU/L | −0.1 | −2.82 | −3.16 | −7.25* |
| Alanine Aminotransferase (ALT) s U/L | −0.11 | −2.5 | −2.7 | −8.3 |
| Aspartate Aminotransferase (AST) s U/L | 1.29 | −5.3 | −5.5 | −13.0 |
| Gamma Glutamyl Transferase (GGT) s U/L | −2.3 | −1.3 | −2.5 | −6.13 |
| Neutrophil to Lymphocyte Ration K/uL | 0.038 | −0.40** | −0.52*** | −0.32* |
| CCL2 (Monocyte Chemotactic Protein-1) pg/mL | −49.3 | −103.05 | −95.53 | −179.0 |
| CCL3 (Macrophage Inflammatory Protein-1 | −1.0 | −7.82*** | −7.14*** | −8.0*** |
| Alpha) pg/mL | | | | |
| CCL5 (RANTES) | −0.35 | −7.0 | −7.8 | −25.3 |
| CCL11 (Eosinophil Chemotactic Protein) pg/mL | −43.75 | −22.77 | −15.21 | −16.25 |
| CCL18 (Macrophage Inflammatory Protein-4) | 0.46 | −31.3** | −32.1** | −48.0** |
| ng/mL | | | | |
| VCAM (Vascular Cell Adnhesion Molecule) | 10.2 | −93.8*** | −94.2*** | −81.0* |
| ng/mL | | | | |
| IL-1 (Interleukin 1) Beta pg/mL | 0.16 | 0.00 | 0.00 | 0.00 |
| IR-IRA (Interleukin 1 Receptor Antagonist) | 17.04 | −28.36* | −34.79** | −26.25 |
| pg/mL | | | | |
| IL-6 (Interleukin 6) pg/mL | 0.19 | −0.28 | −0.44 | −0.30 |
| IL-8 (Interleukin 8) pg/mL | −0.071 | −2.80** | −12.6*** | −4.25** |
| TNF Receptor 2 (STNFR-2) ng/mL | 0.82 | −2.2*** | −2.0*** | −2.7*** |
| TIMP-1 (Tissue Inhibitor Metalloproteinases 1) | −0.25 | −18.27* | −17.26 | −25.3 |
| ng/mL | | | | |
| EN RAGE (Receptor Advanced Glycation End- | 57.75 | −80.09** | −115.50** | −169.0* |
| Products) ng/mL | | | | |
| Apolipoprotein A1S mg/dL | −3.2 | 7.4 | 9.2 | 2.3 |
| High Density Lipoprotein (HDL) U/L | −1.1 | 2.05 | 2.0 | 0.4 |
| LDL/HDL U/L | 0.72 | 0.09 | 0.11 | 0.095 |

*p < 0.05,
**p < 0.01,
***p < 0.001

Additional data with separate measurements for patients concomitantly administered the placebo semaglutide is presented in Table 2. Favorable increases in apolipoprotein Als and HIDL were observed in patients administered 350 mg leronlimab, but not in those administered semaglutide+placebo or in the placebo group. Reductions in inflammation biomarkers IL-1 RA, IL-6, and sTNFR-2 were observed in the group administered 350 mg leronlimab, but not in the group administered semaglutide+placebo or in the placebo group. Levels of the biomarkers CCL5, CCL11, CCL18, VCAM, CCL2, CCL3, and VEGF were also reduced in patients who received 350 mg leronlimab as compared to the placebo group. These markers were increases of less pronounced in the group receiving semaglutide+placebo as compared to the placebo group.

TABLE 2
350 mg Leronlimab vs. Placebo and Placebo + Semaglutide

| | | Placebo + | 350 mg |
|---|---|---|---|
| | Placebo | Semaglutide | leronlimab |
| Biomarker (Baseline to Week 14) | (n = 28) | (n = 5) | (n = 22) |
| MRI PDFF % | 9.80 | −1.85 | −5.94** |
| MRI cT1 | 27.64 | −4.40 | −24.38* |
| Alanine Aminotransferase (ALT) s U/L | −0.11 | −3.6 | −2.5 |
| Aspartate Aminotransferase (AST) s U/L | 1.29 | −2.8 | −5.3 |
| Apolipoprotein A1 s mg/dL | −3.18 | −4.0 | 7.36 |
| High Density Lipoprotein Cholesterol (HDL) | −1.07 | −1.0 | 2.05 |
| mg/dL | | | |
| LDL/HDL Cholesterol Ratio mg/dL | 0.07 | −0.07 | 0.09 |
| Alpha-2-Mactoglobulin s mg/mL | −5.96 | −11.6 | 8.96 |
| CCL2 (Monocyte Chemotactic Protein-1) pg/mL | −49.3 | −6.00 | −103.05 |
| CCL3 (Macrophage Inflammatory Protein-1 | −1.0 | −1.2 | −7.82*** |
| Alpha) pg/mL | | | |
| CCL5 (RANTES) | −0.35 | 1.2 | −7.0 |
| CCL11 (Eosinophil Chemotactic Protein) pg/mL | −43.75 | 0.20 | −22.77 |
| CCL18 (Macrophage Inflammatory Protein-4) | 0.46 | 4.80 | −31.3* |
| ng/mL | | | |
| VEGF (Vascular Endothelian Growth Factor) | −18.86 | −11.2 | −36.23 |
| pg/mL | | | |
| VCAM (Vascular Cell Adnhesion Molecule) | 10.2 | −35.6 | −93.8*** |
| ng/mL | | | |
| IR-IRA (Interleukin 1 Receptor Antagonist) | 17.04 | 26.6 | −28.36* |
| pg/mL | | | |
| IL-6 (Interleukin 6) pg/mL | 0.19 | 0.34 | −0.28 |
| TNF Receptor 2 (STNFR-2) ng/mL | 0.82 | 0.52 | −2.2* |

*p < 0.05,
**p < 0.01,
***p < 0.001

The CCR5 promoter region has been shown to play a critical role in CCR5 transcriptional regulation of disease progression for HIV, HBV, Chagas, heart disease and cancer. CCR5 transcriptional activity and disease progression can be slowed or accelerated. Surface CCR5 expression level also played a part in NASH disease progression and specific CCR5 haplotypes correlated with dose and treatment outcomes. Data for patients with CCR5 haplotypes associated with higher surface expression when administered 700 mg leronlimab is included in FIG. 5 as “700 mg HM” and in FIG. 6A as “700 mg haplotype.”

Specifically, certain haplotypes associated with high cell surface levels of CCR5 on T cells, but poor T cell regulation, were shown to respond differently to leronlimab at 350 mg and 700 mg than patients with certain haplotypes associated with lower cell surface levels of CCR5, but more normal T cell regulation. Patients with haplotypes correlated with increased CCR5 expression required higher doses of leronlimab to benefit from treatment. Specifically, although patients with haplotypes correlated with low to normal surface expression of CCR5 exhibited negative effects when administered 700 mg leronlimab, but benefited from a 350 mg dose, patients with haplotypes correlated with high surface expression of CCR5 exhibited positive effects when administered 700 mg or 350 mg leronlimab, with a greater positive effect at the 700 mg dose. All patients appeared to benefit from a dose that lowered RANTES (CCL5) levels to approximately zero.

Patients with haplotypes associated with increased CCR5 expression exhibited a strong increase in HDL when administered 700 mg leronlimab, as well as reductions in CK18, indicating that CCR5 binding is optimized for surface CCR5 mitochondrial alterations, including reduced apoptosis and necrosis. Improvements at 700 mg leronlimab in Apolipoprotein A1 and steatosis was also observed in these patients. PDFF was decreased by 28%.

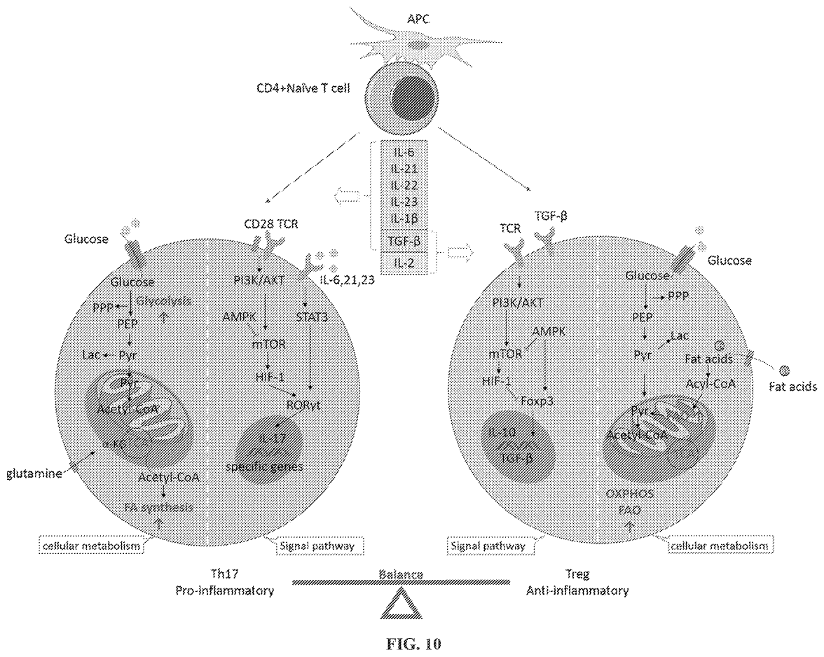

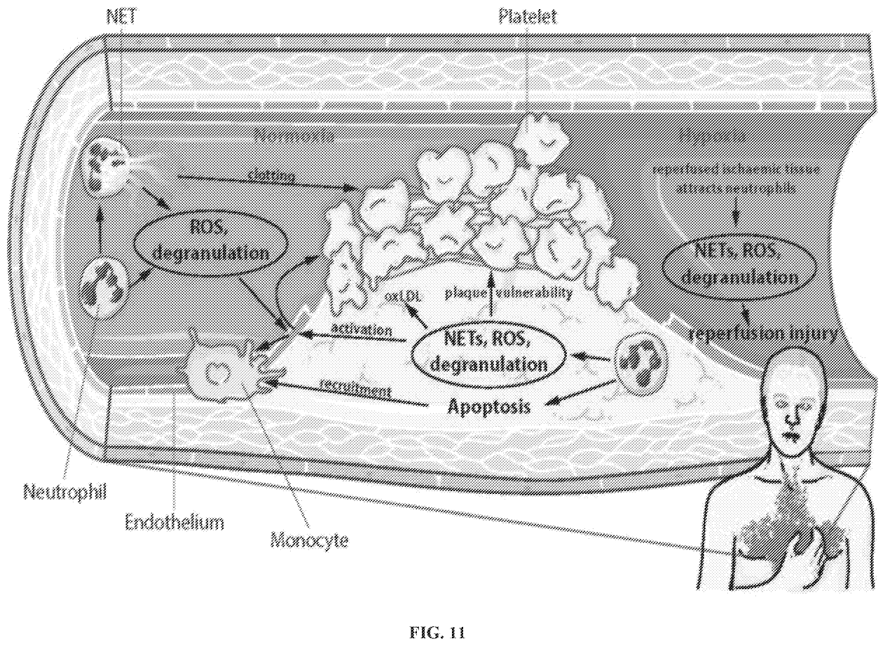

A possible mechanism of action in patients with increased CCR5 cell surface expression is provided in FIG. 10. T helper cells use glycolysis and glutaminolysis for energy, whereas regulatory T cells (T regs) us fatty acids. T regs have a longer life span than T helper cells. Accordingly, new cell produced after administration of leronlimab favor a rebalance of T reg vs. T helper cell levels as newer cells use less glucose and glutamine for energy production. Leronlimab also reduces IL-6 and IL-1 Beta levels so that new CD4 cells used more efficient energy sources, such as fatty acids.

Conversion of the TH response away from TH17/TH1 occurs via reduced TBF beta and reduced IL-6 altering FasL induced mitochrondrial permeabilitization. This in turn alters the Caspase activation extrinsically via T lymphocytes (FasL) to reduce apoptosis and necrosis. Consistent with this, patients exhibiting increased CCR5 cell surface levels who were administered 700 mg leronlimab showed statistically significant reductions for CK18, M30, and M65. from this mechanism and provide further evidence for CCR5 effect on steatosis and metabolism when optimized by haplotype matched dosing.

Insulin resistance may also be improved due to a more predominant TH2/TH22 response and T reg balance as opposed to the higher insulin resistance associated with a predominant TH1/TH17 and Tc (cytotoxic) T cell response.

### CONCLUSIONS

These results demonstrate that leronlimab significantly reduced mean change in PDFF, cT1, CCL3, CCL18, VCAM, and other inflammatory markers from baseline to week 14 for the 350 mg group. Reductions were also observed for the moderate to severe fibro-inflammatory subgroups, where testing for the relevant biomarkers was performed.

The mechanism of action for leronlimab appears to be multifactorial involving VCAM, CCL2, CCL3, CCL11, and CCL18 in addition to competitive binding of CCR5 affecting metabolic and fibroinflammatory parameters with implications for systemic reductions in vascular permeability, arterial stiffness, and oxidative stress.

In particular, CCR5 is believed to dimerize with itself and with other chemokine receptor, and these heterodimers may be capable of signaling via the natural ligands of either receptor. Leronlimab stabilizes CCR5 sell surface levels. The data in this example suggests that CCR5/CCR2 heterodimerization causes reductions in CCL2 and CCL18 in the serum, while reduction in serum CCL3 and CCL11 are likely due to leronlimab-induced CCR5 competitive antagonistic effects. This pathway and effects are illustrated in FIG. 7.

The observed reductions in serum VCAM levels in patients administered 350 mg leronlimab are likely unique to leronlimab and differentiate the effects of the antibody from other CCR2/CCR5 agents. The observed reductions in CCL2 are also likely unique to leronlimab and differentiate leronlimab from other CCR5 antagonists.

The data also suggest that inflammatory control may be needed in addition to metabolism control when administering GLP-1 agents to treat NASH.

The data also suggest that CCR5 haplotype may be considered in determining the appropriate dose of leronlimab for patients with NASH or other inflammatory diseases, with a higher dose being prescribed to those patients with haplotypes causing greater transcription and cell surface expression of CCR5.

The data also suggest that, where patient haplotype is not measured, leronlimab dose may be adjusted to that which achieves a RANTES level of approximately zero, or a level of another biomarker similarly associated with decrease of inflammation.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/354,664, filed Jun. 22, 2022, are incorporated herein by reference, in their entirety. Aspects of the embodiments can be modified, if necessary to employ concepts of the various patents, applications and publications to provide yet further embodiments.

1. A method of treating or preventing nonalcoholic fatty liver disease (NAFLD) or nonalcoholic steatohepatitis (NASH) in a patient by administering to the patient an amount of leronlimab effective to treat or prevent NAFLD or NASH.

2. The method claim 1, wherein the leronlimab is administered by injection.

3. The method of claim 1, wherein the leronlimab is administered weekly.

4. The method of claim 1, wherein the leronlimab is administered in an amount effective to also treat or prevent NASH-related liver fibrosis.

5. The method of claim 1, wherein the leronlimab is administered in a 350 mg dose.

6. The method of claim 1, wherein the leronlimab is administered in a 525 mg dose.

7. The method of claim 1, wherein the leronlimab is administered in a 700 mg dose.

8. The method of claim 1, wherein the patient is evaluated for CCR5 haplotype, and, if the patient has a CCR5 haplotype not associated with increased CCR5 cell surface expression, the patient is administered 350 mg of leronlimab weekly.

9. The method of claim 1, wherein the patient is evaluated for CCR5 haplotype, and, if the patient has a CCR5 haplotype not associated with increased CCR5 cell surface expression, the patient is administered 525 mg of leronlimab weekly.

10. The method of claim 8, wherein the CCR5 haplotype not associated with increased CCR5 cell surface expression does not comprise HHE or HHG.

11. The method of claim 1, wherein the patient is evaluated for CCR5 haplotye, and, if the patient has a CCR5 haplotye associated with increased CCR5 cell surface expression, the patient is administered 525 mg of leronlimab weekly.

12. The method of claim 1, wherein the patient is evaluated for CCR5 haplotype, and, if the patient has a CCR5 haplotype associated with increased CCR5 cell surface expression, the patient is administered 700 mg of leronlimab weekly.

13. The method of claim 11, wherein the CCR5 haplotype associated with increased CCR5 cell surface expression comprises HHE or HHG.

14. The method of claim 1, wherein, subsequent to administration of at least one dose of leronlimab, the level of a biomarker indicating liver function or inflammation is measured, and the dose or dosing frequency of leronlimab is adjusted if the level is not changed by a preset amount as compared to a baseline level or prior level, or if the level is not above or below a preset value.

15. The method of claim 14, wherein the biomarker is one or more of RANTES, CCL2, CCL3, CCL11, CCL18, VCAM, and EN RAGE.

16. The method of claim 15, wherein the biomarker is RANTES and, if the serum level is not sufficiently low to have no clinical inflammatory effect on NASH, the dose or frequency of administration of leronlimab is increased

17. The method of claim 16, wherein the dose is increased from 350 mg weekly to 700 mg weekly.

18. The method of claim 1, wherein, subsequent to administration of at least one dose of leronlimab, the level of an MRI indicator of NASH is measured, and the dose or dosing frequency of leronlimab is adjusted if the level is not changed by a preset amount as compared to a baseline level or prior level, or if the level is not above or below a preset value.

19. The method of claim 18, wherein the MRI indicator of NASH is PDFF or cT1.

20. The method of claim 19, wherein, if the PDFF or cT1 is not below a preset level or decreased as compared to a prior measurement for the patient, the dose of leronlimab is increased from 350 mg weekly to 700 mg weekly.
